(19) United States
(12) Patent Application Publication
(10) Pub. No.: US 2009/0070890 A1
(43) Pub. Date: Mar. 12, 2009

(54) Product

(75) Inventor: Marike Stassar, Baerums Verk (NO)

Correspondence Address: ROTHWELL, FIGG, ERNST & MANBECK, P.C., 1425 K STREET, N.W., SUITE 800, WASHINGTON, DC, 20005, US

(73) Assignee: AFFITECH AS, Oslo (NO)

(21) Appl. No.: 12/055,743

(22) Filed: Mar. 26, 2008

Related U.S. Application Data
(60) Provisional application No. 60/907,241, filed on Mar. 26, 2007.

(30) Foreign Application Priority Data
Mar. 26, 2007 (GB) 0705775.5

(51) Int. Cl. A01K 67/027; C07K 16/00; C07K 16/28; C07K 16/46; C12N 9/10; A61K 39/44; G01N 33/536; A61K 38/21; G01N 33/567; C12P 21/02; A61K 49/00; A61K 39/395; C07H 21/00; C12N 15/63; C12N 5/16

(52) U.S. Cl. 800/13; 530/387.1; 530/387.3; 530/388.1; 530/389.6; 530/391.3; 530/359; 435/193; 530/391.7; 536/23.53; 536/23.4; 435/320.1; 435/325; 424/130.1; 424/178.1; 424/9.1; 436/536; 435/69.1; 424/85.4; 435/7.21

(57) ABSTRACT

The invention relates to tumor-specific binding proteins and all uses thereof. In particular, the invention relates to antibodies or antibody fragments specific for antigens or molecules on cancer cells (CD166) and to methods of use thereof. Binding proteins comprising specific heavy and light chain CDRs are disclosed wherein the binding protein shows a measurable or significant binding to breast cancer cell line MDA-MB 231 but shows insignificant or unmeasurable binding to granulocytes or peripheral blood lymphocytes (PBLs).

This application claims priority from U.S. provisional application Ser. No. 60/907,241, filed Mar. 26, 2007. This prior application is incorporated herein by reference.

The invention relates to tumor-specific binding proteins and all uses thereof. In particular, the invention relates to antibodies or antibody fragments specific for antigens or molecules on cancer cells and to methods of use thereof. Preferably, the antigen is CD166.

In the year 2000, an estimated 22 million people were suffering from cancer worldwide and 6.2 millions deaths were attributed to this class of diseases. Every year, there are over 10 million new cases and this estimate is expected to grow by 50% over the next 15 years (WHO, World Cancer Report. Bernard W. Stewart and Paul Kleihues, eds. IARC Press, Lyon, 2003).

Mamma carcinoma is a carcinoma of breast tissue. Worldwide it is the most common form of cancer in women, affecting approximately 10% of all females at some stage of their life (in the Western world). Although significant efforts have been made to achieve early detection and effective treatment, about 20% of all women with breast cancer still die from the disease. Mamma carcinoma is the second most common cause of cancer deaths in women.

Current cancer treatments are limited to invasive surgery, radiation therapy and chemotherapy, all of which cause either potentially severe side-effects, non-specific toxicity and/or traumatizing changes to ones body image and/or quality of life. Cancer can become refractory to chemotherapy reducing further treatment options and likelihood of success. Some cancers with a relatively high treatment success rate, such as breast cancer, also have a very high incidence rate and, thus, remain major killers.

For instance, according to WHO, there are over 1.2 million new cases of breast cancer, worldwide, each year. The mainstay of breast cancer treatment is surgery (lumpectomy and mastectomy) when the tumor is localized, with possible adjuvant hormonal therapy (classically with tamoxifen or aromatase inhibitors). Chemotherapy and radiotherapy are used, either to support surgery or in cases of disseminated disease. Recently, the use of the HER-2/neu specific humanized monoclonal antibody Herceptin showed very promising results.

There are several prognostic factors associated with breast cancer. Stage is the single most important prognostic factor in breast cancer, as it takes into consideration local involvement, lymph-node status and presence of metastases. The higher the stage at the time of diagnosis, the worse the prognosis. Presence of estrogen and progesterone receptors in the cancer cell is the other important classical prognostic factor. In addition, hormone receptor positive breast cancer is usually associated with much better prognosis compared to hormone negative breast cancer. Also, more recently, HER-2/neu status has been described as a prognostic factor. Prognosis for localized disease is relatively good with a 5 years survival rate of around 50% but once the cancer has metastasized, it is incurable with an average survival of around 2 years. Despite improving treatment success rates, nearly 400,000 women die of breast cancer each year, the highest number of deaths to cancer in woman, ahead of deaths to lung cancer. Among the short and long term survivors, most will suffer the life-long trauma of invasive and disfiguring surgical treatment.

There are many more examples of cancer where current treatments do not meet the needs of patients either due to their lack of efficacy and/or because they have high morbidity rates and severe side-effects. Those selected statistics and facts however, illustrate well the need for cancer treatments with better safety and efficacy profiles.

One of the causes for the inadequacy of current cancer treatments is their lack of selectivity for affected tissues and cells. Surgical resection always involves the removal of apparently normal tissue as a “safety margin” which can increase morbidity and risk of complications. It also always removes some of the healthy tissue that may be interspersed with tumor cells and that could potentially maintain or restore the function of the affected organ or tissue. Radiation and chemotherapy will kill or damage many normal cells due to their non-specific mode of action. This can result in serious side-effects such as severe nausea, weight loss and reduced stamina, loss of hair etc., as well as increasing the risk of developing secondary cancer later in life. Treatment with greater selectivity for cancer cells would leave normal cells unharmed thus improving outcome, side-effect profile and quality of life.

The selectivity of cancer treatment can be improved by using antibodies that are specific for molecules present only or mostly on cancer cells or which are present in higher levels on cancer cells or overexpressed in cancer cells. Such antibodies can be used to modulate the immune system and enhance the recognition and destruction of the cancer by the patient's own immune system. Most antibodies tested to date have been raised against known cancer markers in the form of mouse monoclonal antibodies, sometimes “humanized” through molecular engineering. Unfortunately, their targets can also be present in significant quantities on a subset of normal cells thus raising the risk of non-specific toxic effects. Furthermore, these antibodies are mouse proteins that are being seen by the human patient's immune system as foreign proteins. The ensuing immune reaction and antibody response can result in a loss of efficacy or in side-effects.

The inventors have used a different approach in their development of antibodies for cancer treatment, in particular breast cancer treatment. Instead of immunizing experimental animals with cancer cells or isolated cancer cell markers, they have sought out to identify only those markers that are recognized by the human immune system as sufficiently foreign to allow for specific antibodies against them to be found. This implies that the markers or antigens are less abundant or absent on normal cells and, thus, the risk of non-specific toxicity is further reduced. Thus, antibodies showing high selectivity for cancer cells/tumor cells, in particular breast cancer cells, over normal cells have been identified. Such selective antibodies are the subject of this patent application. In addition to being selective, preferably such antibodies are fully compatible with the patient's immune system by virtue of being fully-human proteins. The antibodies of the invention can be used for diagnostic or therapeutic uses (in particular for cancer, especially breast cancer) or as a basis for engineering other binding molecules for the target antigen. The antibodies can also be used to isolate and identify the molecule to which they bind. The role of the antigen in cancer can then be studied or the antigen can be used to develop other cancer treatments. The inventors have determined the identity of the antigen to which the antibodies of the invention bind.

The antigen is CD166, also known as ALCAM (activated leukocyte cell adhesion molecule), MEMD, SB-10, KG-CAM and neurolin. CD166 is a 105-kDa transmembrane glycoprotein which is a member of the immunoglobulin superfamily with five extracellular immunoglobulin-like domains. It has a short cytoplasmic tail and its extracellular part comprises five Ig domains: two amino-terminal variable (V) type Ig domains followed by three constant (C) type Ig domains (V₁V₂C₁C₂C₃). A preferred epitope of the antibodies of the invention is located on the extracellular domain of the CD166 antigen.

Thus, CD166 is a cell surface protein. It can be found on activated leucocytes, cortical and medullary thymic fibroblasts, some subsets of neural cells and some epithelial cells. As a general rule, ALCAM is often found on cells migrating/and or in dynamic growth.

CD166 was first identified as a CD6 ligand, but it also mediates homophilic CD166 interactions. It is expressed developmentally in cells of all 3 embryonic lineages, but its expression is believed to be limited to subsets of cells in most adult tissues. CD166 is thought to be localized at intercellular junctions in epithelium and this may be as part of the adhesive complex that maintains tissue architecture. CD166 has been linked with cell migration, cancer development and with cancer progression and metastasis. Aberrant expression of CD166 has been implicated in several human tumors including melanoma, prostate cancer, breast cancer, colorectal carcinoma, bladder cancer, ovarian cancer, pancreas cancer, lung cancer and esophageal squamous cell carcinoma.

The present inventors have prepared human tumor-specific antibodies that bind to cancer cells, in particular breast tumor cells. Importantly, the antibodies do not significantly bind to normal cells making them suitable candidates for diagnostics and tumor therapy, preferably breast cancer therapy. Other preferred cancers are described elsewhere herein.

The inventors have cloned and sequenced the antibodies and determined the sequence of the antibody light and heavy chain variable regions, including the complementarity determining regions (CDRs) 1, 2 and 3.

Accordingly, the present invention provides tumor-specific binding proteins, e.g. antibody molecules. In a preferred embodiment the invention provides human binding proteins, e.g. antibody molecules, with these properties.

The term “normal cells” is used herein to refer to non-cancerous cells. This term encompasses healthy cells which occur naturally within the human body, in particular peripheral red blood cells or granulocytes. Thus, preferably the binding protein does not significantly bind to peripheral blood lymphocytes (PBLs) and/or does not significantly bind to granulocytes. In a preferred embodiment, the binding protein shows a measurable or significant binding to breast cancer cell line MDA-MB 231 but shows insignificant or unmeasurable binding to granulocytes or peripheral blood lymphocytes (PBLs).

The term “do not significantly bind to normal cells” should be understood such that any binding of the binding protein to normal cells does not prohibit the use of said binding protein for therapeutic or diagnostic purposes. Thus, by “insignificant” binding to normal cells is preferably meant that the binding of the binding protein to normal cells is weaker than its binding to one or more tumor cells. For therapeutic purposes the main consideration is that the binding protein must bind more strongly to one or more types of tumor cells than to any healthy cells (or one or more corresponding healthy cells) with which the binding protein may come into contact during the therapeutic application or that any binding to normal cells and the potentially negative effects caused by that binding are outweighed by the positive effects in the treatment of cancer achieved by the binding of the antibody to cancer cells.

For diagnostic purposes the main consideration is that the binding of the binding protein (preferably antibody) to tumor cells generates a signal clear enough to allow a diagnosis of the cancer.

The term “tumor specific” should be interpreted such that the binding of the binding protein to the tumor cells is specific enough to allow the use of said binding protein for therapeutic or diagnostic purposes. The skilled person can easily determine if any given binding protein is tumour specific by comparing the binding strength to the target tumor cell with the binding strength to one or more types of normal cells, e.g. peripheral red blood cells or granulocytes. Any reference herein to “tumor” or “cancer” should be understood to include a reference to “breast tumors” and “breast cancer”, to “prostate tumor” and “prostate cancer”, to “ovarian tumor” and “ovarian cancer”, to “colon tumor” and “colon cancer”, to “lung tumor” and “lung cancer”, to “kidney tumor” and “kidney cancer” and/or to “brain tumor” and “brain cancer”, preferably to “breast tumors” and “breast cancer”.

The binding proteins of the invention are tumor specific in that the binding proteins bind to one or more types of tumor cell, preferably breast cancer cells, but the binding to one or more types of normal cells is insignificant or not prohibitive for diagnostic or therapeutic applications. For example, the binding protein may bind to normal tissue which will never come into contact with the binding proteins of the invention, e.g. normal tissue in the brain, which the binding proteins will not reach if they are not administered to the brain because they can not cross the blood brain barrier. In some embodiments, the binding protein may also show significant binding to some types of normal, i.e. healthy cells, which are not essential or not existing in the patient to be treated (such as prostate cells in case of treating female patients, or treatment of male patients for metastasis of prostate cancer after removal of the organ) and/or which can be regenerated quickly (such as epithelial cells in the intestine), but binding to other types of normal cells is insignificant. In these embodiments, the binding proteins may be used for therapeutic applications because the binding proteins bind to tumor cells and most or all essential normal cells are substantially not affected by the binding protein.

Preferably, the binding proteins bind to one or more types of tumor cells, preferably breast cancer cells, in a way that or at a level that is effective for diagnostic or therapeutic purposes (e.g. show significant and measurable binding to one or more types of tumor cells, preferably breast cancer cells).

Preferably, the binding proteins have a binding affinity for one or more types of cancer cells which corresponds to a Km of less than 1 μM, more preferably of less than 500, 400 or 300 nM, even more preferably of less than 200, 190, 180, 170, 160, 150, 140, 130, 120, 110, or 100 nM, most preferably of less than 90, 80, 70, 60, 50, 45, 40, 35, 30, 25, 20, 15, 10, 5 or 1 nM. For example, the binding affinity may be 1.7×10⁻⁷ M or less, or 3×10⁻⁸M or less, or 2.9×10⁻⁸M or less, or 2.1×10⁻⁸ M or less, or 1×10⁻⁸M or less. Binding affinities can also be referred to in terms of K_{d}, in which case similar values are appropriate. Binding affinities can be measured using any appropriate format of binding protein of the invention. Where the binding proteins are antibodies or antibody fragments preferred formats are whole antibodies (e.g. IgG), scFv or Fab. Any appropriate method of determining Km or K_{d} may be used. However, preferably the Km or K_{d} is determined by testing various concentrations of the binding protein against a fixed number of target cells (or target antigen) in vitro to establish a saturation curve, for example using the Lineweaver-Burk method or a BiaCore apparatus and appropriate software, such as the 1:1 binding model in the BiaCore 3000 Evaluation software. Suitable assays are described in Example 3 for illustrative purposes.

The binding proteins preferably have a Km or K_{d} for one or more types of tumor cells which is at least 50% less, more preferably at least 1, 2, 3, 4 or 5 orders of magnitude lower than the Km or K_{d} for one or more types of non-cancerous or normal cells, e.g. PBL cells or granulocytes, when binding affinity is assayed under comparable conditions, in particular using the same dosage of binding protein and cells in each assay.

The binding proteins of the present invention can preferably bind to CD166 or fragments of CD166, in particular fragments comprising or consisting of the extracellular domains of CD166, or entities comprising CD166 or fragments of CD166, or can inhibit or significantly reduce the function of CD166 or prevent CD166 interacting with its natural ligands. The present invention thus further provides binding proteins, e.g. antibody molecules, that can act as modulators of CD6 or CD166. Such modulators might be antagonists. Alternatively, the present invention can further provide binding proteins, e.g. antibody molecules, that can act as agonists of CD6 or CD166.

Preferred binding proteins of the invention can bind to human CD166 (or fragments thereof) and preferably also to murine CD166 (or fragments thereof). Exemplary and preferred binding affinities of the antibodies of the invention to CD166 are as described above for the binding affinities to tumor cells.

Amino acid and/or DNA sequences of antibody molecules which can specifically bind to tumor cells, preferably breast cancer cells, and which can preferably bind to CD166, their V_{H} and V_{L} domains including complementarity determining regions (CDRs) are set forth in the various SEQ ID Nos. listed herein.

In one embodiment the present invention provides a binding protein comprising a heavy chain CDR3 domain comprising the amino acid sequence of SEQ ID No: 5 or a sequence substantially homologous thereto.

Alternatively or in addition, the binding protein comprises a heavy chain CDR2 domain comprising the amino acid sequence of SEQ ID No.4 or a sequence substantially homologous thereto.

Alternatively or in addition, the binding protein comprises a heavy chain CDR 1 domain comprising the amino acid sequence of SEQ ID No: 3 or a sequence substantially homologous thereto.

Alternatively or in addition, the binding protein comprises a light chain CDR 1 domain comprising the amino acid sequence of SEQ ID No: 6 or a sequence substantially homologous to said sequences.

Alternatively or in addition, the binding protein comprises a light chain CDR 2 domain comprising the amino acid sequence of SEQ ID No: 7 or a sequence substantially homologous to said sequences.

Alternatively or in addition, the binding protein comprises a light chain CDR 3 domain comprising the amino acid sequence of SEQ ID No: 8 or a sequence substantially homologous to said sequences.

Thus, the binding protein of the present invention may comprise one or more of the following in any combination: a heavy chain CDR1 comprising the amino acid sequence of SEQ ID No: 3; a heavy chain CDR2 comprising the amino acid sequence of SEQ ID No: 4; a heavy chain CDR3 comprising the amino acid sequence of SEQ ID No: 5; a light chain CDR1 comprising the amino acid sequence of SEQ ID No: 6; a light chain CDR2 comprising the amino acid sequence of SEQ ID No: 7; and/or a light chain CDR3 comprising the amino acid sequence of SEQ ID No: 8, or a sequence substantially homologous to any of the foregoing SEQ ID Nos.

Preferably, the binding protein comprises both a heavy chain CDR 3 as defined above and a light chain CDR3 as defined above. More preferably, one or more of the above defined CDR1 and CDR 2 domains are also present.

In one preferred embodiment, the heavy chain CDR1 comprising the amino acid sequence of SEQ ID No: 3 or a sequence substantially homologous thereto, CDR2 comprising the amino acid sequence of SEQ ID No: 4 or a sequence substantially homologous thereto and CDR3 comprising the amino acid sequence of SEQ ID No: 5, or a sequence substantially homologous to any one of the foregoing SEQ ID Nos, are present individually or in combination.

In another preferred embodiment, the light chain CDR1 comprising the amino acid sequence of SEQ ID No: 6 or a sequence substantially homologous thereto, CDR2 comprising the amino acid sequence of SEQ ID No: 7 or a sequence substantially homologous thereto and CDR3 comprising the amino acid sequence of SEQ ID No: 8 or a sequence substantially homologous to any one of the foregoing SEQ ID Nos, are present individually or in combination.

Viewed alternatively, the present invention provides a binding protein comprising a heavy chain CDR3 domain and/or a light chain CDR3 domain as defined above. Said binding protein optionally further comprises a heavy chain CDR2 domain and/or a light chain CDR2 domain as defined above and/or further comprises a heavy chain CDR1 domain and/or a light chain CDR1 domain as defined above.

Viewed yet alternatively, the present invention provides a binding protein comprising a heavy chain CDR1 domain and/or a light chain CDR1 domain as defined above. Said binding protein optionally further comprises a heavy chain CDR3 domain and/or a light chain CDR3 domain as defined above and/or further comprises a heavy chain CDR2 domain and/or a light chain CDR2 domain as defined above.

Viewed yet alternatively, the present invention provides a binding protein comprising a heavy chain CDR2 domain and/or a light chain CDR1 domain as defined above. Said binding protein optionally further comprises a heavy chain CDR3 domain and/or a light chain CDR3 domain as defined above and/or further comprises a heavy chain CDR1 domain and/or a light chain CDR1 domain as defined above.

A yet further embodiment of the invention provides a binding protein comprising one or more of the CDRs of the invention or sequences substantially homologous thereto as set out herein. Preferred binding proteins comprise one or more of the CDRs selected from the group consisting of SEQ ID NOs. 3, 4, 5, 6, 7 and 8 or a sequence substantially homologous to any one of the foregoing SEQ ID Nos.

Thus, in preferred embodiments the binding protein comprises a light chain CDR 1 comprising the amino acid sequence RASQDISSYFA (SEQ ID NO. 6), or a sequence substantially homologous thereto; and/or comprises a light chain CDR 2 comprising the amino acid sequence AASTLRS (SEQ ID NO:7), or sequences substantially homologous thereto; or comprises a light chain CDR3 comprising the amino acid sequence QQSYSTPRIT (SEQ ID NO: 8), or sequences substantially homologous thereto.

Preferred binding proteins comprise two or more of the light chain CDRs of the invention or sequences substantially homologous thereto as described above. Especially preferred binding molecules comprise 3 of the light chain CDRs of the invention or sequences substantially homologous thereto as described above (i.e. one of each of the light chain CDR1 and CDR2 and CDR3).

Other preferred binding proteins comprise two or more of the heavy chain CDRs of the invention or sequences substantially homologous thereto as described above. Especially preferred binding molecules comprise 3 of the heavy chain CDRs of the invention or sequences substantially homologous thereto as described above (i.e. one of each of the heavy chain CDR1 and CDR2 and CDR3). Most preferred binding proteins comprise 3 of the light chain CDRs of the invention or sequences substantially homologous thereto as described above and 3 of the heavy chain CDRs of the invention or sequences substantially homologous thereto as described above.

Especially preferred binding molecules comprise a heavy chain CDR1 domain of SEQ ID NO:3, a heavy chain CDR2 domain of SEQ ID NO: 4, and a heavy chain CDR3 domain of SEQ ID NO: 5, or sequences substantially homologous thereto; and/or comprise a light chain CDR1 domain of SEQ ID NO: 6, a light chain CDR2 domain of SEQ ID NO: 7, and a light chain CDR 3 domain of SEQ ID NO: 8, or sequences substantially homologous thereto.

Further preferred embodiments provide binding proteins comprising a V_{H} domain which comprises one or more of the heavy chain CDRs of the invention or sequences substantially homologous thereto, as described above, and/or a V_{L} domain which comprises one or more of the light chain CDRs of the invention or sequences substantially homologous thereto, as described above.

Preferred light chain variable regions (V_{L} domains) comprise 2 or more of the light chain CDRs of the invention or sequences substantially homologous thereto, as described above. Especially preferred V_{L} domains comprise 3 of the light chain CDRs of the invention or sequences substantially homologous thereto as described above (i.e. one of each of CDR1, CDR2 and CDR3). Preferred heavy chain variable regions (V_{H} domains) comprise 2 or more of the heavy chain CDRs of the invention or sequences substantially homologous thereto, as described above. Especially preferred V_{H} domains comprise 3 of the heavy chain CDRs of the invention or sequences substantially homologous thereto as described above (i.e. one of each of CDR1, CDR2 and CDR3). Most preferred binding proteins comprise 3 of the light chain CDRs of the invention or sequences substantially homologous thereto as described above and 3 of the heavy chain CDRs of the invention or sequences substantially homologous thereto as described above.

Preferred CDR domains and combinations thereof comprising the V_{H} or V_{L} domains are described elsewhere herein. However, an especially preferred V_{H} domain (or binding protein) comprises the CDR3 of GGGVVEF (SEQ ID NO:5), or sequences substantially homologous thereto and an especially preferred V_{L} domain (or binding protein) comprises the CDR3 of QQSYSTPRIT (SEQ ID NO. 8), or sequences substantially homologous thereto.

In a further embodiment, the V_{L} domain (or binding protein) comprises the CDR regions of RASQDISSYFA (CDR 1) (SEQ ID NO:6) and/or AASTLRS (CDR 2) (SEQ ID NO: 7) and/or QQSYSTPRIT (CDR 3) (SEQ ID NO: 8), or sequences substantially homologous thereto.

In a further embodiment, the V_{H} domain (or binding protein) comprises the CDR regions of SYAMS (CDR 1) (SEQ ID NO: 3) and/or AISGSGGSTYYADSVKG (CDR 2) (SEQ ID NO: 4) and/or GGGWEF (CDR 3) (SEQ ID NO: 5), or sequences substantially homologous to any of the aforementioned sequences.

Any combination of the above discussed V_{L} and V_{H} domains can be present in the binding proteins of the invention.

Preferred embodiments of the invention provide a binding protein comprising a V_{H} domain which has the amino acid sequence of SEQ ID NO. 9 or sequences substantially homologous thereto and/or a V_{L} domain which has the amino acid sequence of SEQ ID NO. 10, or sequences substantially homologous thereto.

In a yet further embodiment the present invention provides a binding protein comprising the amino acid sequence of SEQ ID No. 2 (also referred to herein as clone EJ212/007-Cl2-5, scFv), or comprising a fragment thereof, or a sequence substantially homologous thereto.

A yet further embodiment of the present invention provides an IgG form of EJ212/007-Cl2-5, the heavy chain of which comprises a variable region which comprises the amino acid sequence of SEQ ID NO:9 or a sequence substantially homologous thereto, and a heavy chain constant region, and the light chain of which comprises a variable region which comprises the amino acid sequence of SEQ ID NO:10 or a sequence substantially homologous thereto, and a light chain constant region.

A yet further embodiment of the present invention provides an IgG form of EJ212/007-Cl2-5, the heavy chain of which comprises a variable region which comprises the amino acid sequence of SEQ ID NO:9 or a sequence substantially homologous thereto, and a constant region which comprises the amino acid sequence of SEQ ID NO:12 or a sequence substantially homologous thereto, and the light chain of which comprises a variable region which comprises the amino acid sequence of SEQ ID NO:10 or a sequence substantially homologous thereto, and a constant region which comprises the amino acid sequence of SEQ ID NO:13 or a sequence substantially homologous thereto.

The term “binding protein” as used herein refers to proteins that specifically bind to another substance. In particular, binding proteins of the invention can preferably specifically bind to CD166 or fragments of CD166, in particular fragments comprising or consisting of the extracellular domains of CD166, or entities comprising CD166 or fragments of CD166, or can inhibit or significantly reduce the function of CD166 or prevent CD166 interacting with its natural ligands. In a preferred embodiment binding proteins are human proteins. In a further preferred embodiment, binding proteins are antibodies or antibody fragments or comprise antibodies or antibody fragments. Human antibodies or human antibody fragments are especially preferred.

Preferred antibodies of the invention comprise at least one heavy chain variable region that comprises three CDRs and at least one light chain variable region that comprises three CDRs. Exemplary and preferred sequences for these CDRs are described herein. However, although preferred binding proteins of the invention comprise CDRs of the invention, it should be noted that the binding proteins of the invention can also comprise one or more CDRs of the invention in combination with other CDRs not of the invention, provided that the tumor specific properties, and preferably the CD166 binding properties, of the binding proteins of the invention as outlined above are still present.

However, it is also well documented in the art that the presence of three CDRs from the light chain variable domain and three CDRs from the heavy chain variable domain of an antibody is not necessary for antigen binding.

For example, camelid antibodies (Hamers-Casterman et al., 1993, Nature, 363(6428):446-448; Arbabi Ghahroudi et al., 1997, FEBS Lett., 414:521-526) have an extensive antigen binding repertoire but are devoid of light chains. Also, results with single domain antibodies comprising VH domains alone (Ward et al., 1989, Nature, 341 (6242):544-546; Davies and Riechmann, 1995, Biotechnology (NY), 13:475-479) or VL domains alone (van den Beucken et al., 2001, J. Mol. Biol., 310:591-601) show that these domains can bind to antigen with acceptably high affinities. Thus, three CDRs can effectively bind antigen.

It is also known that a single CDR, or two CDRs, can effectively bind antigen. As a first example, a single CDR can be inserted into a heterologous protein and confer antigen binding ability on the heterologous protein, as exemplified by showing that a VH CDR3 region inserted into a heterologous protein, such as GFP, confers antigen binding ability on the heterologous protein (Kiss et al., 2006, Nucleic Acids Research, 34(19):e132; Nicaise et al., 2004, Protein Sci., 13: 1882-1891).

It is further known that two CDRs can effectively bind antigen, and even confer superior properties than possessed by the parent antibody. For example, it has been shown (Qiu et al., 2007, Nature Biotechnology, 25(8): 921-929) that two CDRs from a parent antibody (a VH CDR1 and a VL CDR3 region) retain the antigen recognition properties of the parent molecule but have a superior capacity to penetrate tumors. Joining these CDR domains with an appropriate linker sequence (e.g., from VH FR2) to orientate the CDRs in a manner resembling the native parent antibody produced even better antigen recognition. Therefore, it is known in the art that it is possible to construct antigen binding antibody mimetics comprising two CDR domains (preferably one from a VH domain and one from a VL domain, more preferably, with one of the two CDR domains being a CDR3 domain) orientated by means of an appropriate framework region to maintain the conformation found in the parent antibody.

Thus, although preferred antibodies of the invention might comprise six CDR regions (three from a light chain and three from a heavy chain), antibodies with fewer than six CDR regions and as few as one or two CDR regions are encompassed by the invention. In addition, antibodies with CDRs from only the heavy chain or light chain are also contemplated.

The binding proteins of the present invention are preferably tumor specific in that the binding proteins bind to one or more types of tumor cell or sample, but the binding to one or more types of normal cells is insignificant or not prohibitive for diagnostic or therapeutic applications. For example, the binding to one or more types of tumor cell may be stronger than the binding to one or more types of normal cells. Alternatively or in addition, the binding protein may bind to normal tissue which will never come into contact with the binding proteins of the invention, e.g. normal tissue in the brain, which the binding proteins will not reach if they are not administered to the brain because they can not cross the blood brain barrier. Preferably, the binding proteins bind to one or more types of tumor cell or sample, preferably one or more types of breast cancer cell or sample, in a way that or at a level that is effective for diagnostic or therapeutic purposes (e.g. show significant and measurable binding to one or more types of tumor cells or samples, preferably breast cancer cells, but show weaker binding, preferably no significant binding to one or more types of normal cells or samples). Specificity for tumors is particularly desired when it is desired to use the binding proteins of the invention for tumor therapy or diagnosis.

Appropriate ways of assessing such tumor specificity are well known and described in the art, for example by FACS or immunohistochemical profiling, in which generally the binding of a binding protein to one or several tumor cell lines or samples is compared to the binding of the protein to one or several normal cell lines or samples, and the finding of a measurable or significant difference (increase) in binding to tumor versus normal cells indicates tumor specificity. Exemplary normal and tumor cell lines or samples which can be used are well known and described in the art, some of which are described in the Examples (e.g. an appropriate tumor cell line is the breast cancer cell line MDA-MB 231 (ATCC: HTB-26) or the lung carcinoma cell line SW900 (ATCC:HTB-59) and appropriate normal cells are blood cells, e.g. PBLs or granulocytes). For example, preferred binding proteins of the invention can bind (e.g. show measurable or significant binding) to a breast cancer cell line such as MDA-MB 231 but show insignificant or unmeasurable binding to granulocytes or PBLs (peripheral blood lymphocytes). Put another way, such binding proteins show a measurable (and preferably significant) increase in binding to breast cancer cells such as MDA-MB 231 cells or to lung cancer cells such as SW900 cells compared with normal cells such as granulocytes or PBLs.

Preferably the significant difference in binding is statistically significant, preferably with a probability value of <0.1, preferably <0.05, more preferably <0.01. Appropriate methods of determining statistical significance are well known and documented in the art and any of these may be used.

As an alternative example for assessing tumor specificity immunohistochemical profiling can be carried out on normal and tumor tissue samples, e.g. as described in the present Examples. Preferred binding proteins can bind (e.g. show measurable or significant binding) to tumor tissue samples (e.g. breast cancer, ovarian cancer, prostate cancer, kidney cancer or brain cancer samples) but show weaker, preferably insignificant or unmeasurable binding to normal tissues. Other preferred binding proteins can bind to melanoma, prostate cancer, breast cancer, colorectal carcinoma, bladder cancer, ovarian cancer, pancreas cancer, lung cancer or esophageal squamous cell carcinoma. In general, preferred binding proteins can bind to cancer cell types which express the CD166 antigen.

Immunohistochemistry techniques can be used to score the binding of binding proteins to cells or samples, e.g. as described in Example 4 and Table 3. Preferred binding proteins show weak or strong, preferably strong immunohistochemical scores.

The term “human” as used herein in connection with antibody molecules and binding proteins refers to binding proteins having variable (e.g. V_{H}, V_{L}, CDR or FR regions) and/or constant antibody regions derived from or corresponding to sequences found in humans, e.g. in the human germline or somatic cells. The “human” binding proteins of the invention further include amino acid residues not encoded by human sequences, e.g. mutations introduced by random or site directed mutations in vitro (in particular mutations which involve conservative substitutions or mutations in a small number of residues of the binding protein, e.g. in 1, 2, 3, 4 or 5 of the residues of the binding protein, preferably e.g. in 1, 2, 3, 4 or 5 of the residues making up one or more of the CDRs of the binding protein). Thus, the “human” antibodies of the invention include sequences derived from and related to sequences found in humans, but which may not naturally exist within the human antibody germline repertoire in vivo. In addition, the human binding proteins of the present invention include proteins comprising human consensus sequences identified from human sequences, or sequences substantially homologous to human sequences.

In addition, the human binding proteins of the present invention are not limited to combinations of V_{H}, V_{L}, CDR or FR regions which are themselves found in combination in human antibody molecules. Thus, the human binding proteins of the invention can include or correspond to combinations of such regions which do not necessarily exist naturally in humans.

In preferred embodiments, the human antibodies will be fully human antibodies. “Fully human” antibodies, as used herein, are antibodies comprising “human” variable region domains and/or CDRs, as defined above, without substantial non-human antibody sequences or without any non-human antibody sequences. For example, antibodies comprising human variable region domains and/or CDRs “without substantial non-human antibody sequences” are antibodies, domains and/or CDRs in which only about 5, 4, 3, 2 or 1 amino acids are amino acids that are not encoded by human antibody sequences. Thus, “fully human” antibodies are distinguished from “humanized” antibodies, which are based on substantially non-human variable region domains, e.g., mouse variable region domains, in which certain amino acids have been changed to better correspond with the amino acids typically present in human antibodies.

The “fully human” antibodies of the invention may be human variable region domains and/or CDRs without any other substantial antibody sequences, such as being single chain antibodies. Alternatively, the “fully human” antibodies of the invention may be human variable region domains and/or CDRs integral with or operatively attached to one or more human antibody constant regions. Certain preferred fully human antibodies are IgG antibodies with the full complement of IgG constant regions.

In other embodiments, “human” antibodies of the invention will be part-human chimeric antibodies. “Part-human chimeric” antibodies, as used herein, are antibodies comprising “human” variable region domains and/or CDRs operatively attached to, or grafted onto, a constant region of a non-human species, such as rat or mouse. Such part-human chimeric antibodies may be used, for example, in pre-clinical studies, wherein the constant region will preferably be of the same species of animal used in the pre-clinical testing. These part-human chimeric antibodies may also be used, for example, in ex vivo diagnostics, wherein the constant region of the non-human species may provide additional options for antibody detection.

The term “antibody” or “antibody molecule” as used herein refers to immunoglobulin molecules or other molecules which comprise an antigen binding domain.

The term “antibody” or “antibody molecule” as used herein is thus intended to include whole antibodies (e.g. IgG, IgA, IgE, IgM, or IgD, preferably IgG or IgM), monoclonal antibodies, polyclonal antibodies, humanized and chimeric antibodies. Antibody fragments which comprise an antigen binding domain are also included. The term “antibody fragment” as used herein is intended to include any appropriate antibody fragment that displays antigen binding function (preferably CD166 binding function), for example Fab, Fab′, F(ab′)₂, scFv, Fv, dsFv, ds-scFv, Fd, dAbs, T and Abs dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, T and Abs, ds-scFv, dimers, minibodies, diabodies, bispecific antibody fragments and other fragments can also be synthesized by recombinant techniques or can be chemically synthesized. Techniques for producing antibody fragments are well known and described in the art. The present inventors have inter alia prepared scFv, Fab and IgG antibodies having the binding characteristics described herein.

The antibodies or antibody fragments can be produced naturally or can be wholly or partially synthetically produced. Thus the antibody may be from any appropriate source, for example recombinant sources and/or produced in transgenic animals or transgenic plants. Thus, the antibody molecules can be produced in vitro or in vivo.

Preferably the antibody or antibody fragment comprises an antibody light chain variable region (V_{L}) and an antibody heavy chain variable region (V_{H}) which generally comprise the antigen binding site. In certain embodiments, the antibody or antibody fragment comprises all or a portion of a heavy chain constant region, such as an IgG1, IgG2, IgG3, IgG4, IgA1, IgA2, IgE, IgM or IgD constant region. Preferably, the heavy chain constant region is an IgG1 heavy chain constant region, or a portion thereof. Furthermore, the antibody or antibody fragment can comprise all or a portion of a kappa light chain constant region or a lambda light chain constant region, or a portion thereof. Preferably, the light chain constant region is a kappa light chain constant region, or a portion thereof. All or part of such constant regions may be produced naturally or may be wholly or partially synthetic. Appropriate sequences for such constant regions are well known and documented in the art.

The term “fragment” as used herein refers to fragments of biological relevance, e.g. fragments which can contribute to antigen binding, e.g. form part of the antigen binding site, or can contribute to the inhibition or reduction in function of the antigen or can contribute to the prevention of the antigen interacting with its natural ligands. Preferred fragments thus comprise a heavy chain variable region (V_{H} domain) and/or a light chain variable region (V_{L} domain) of the antibodies of the invention. Preferred fragments retain the ability to bind to tumor cells and preferably to bind to the CD166 antigen, as described elsewhere herein (i.e. are antigen binding fragments). Other preferred fragments comprise one or more of the heavy chain complementarity determining regions (CDRs) of the antibodies of the invention (or of the V_{H} domains of the invention), or one or more of the light chain complementarity determining regions (CDRs) of the antibodies of the invention (or of the V_{L} domains of the invention). Preferred fragments are thus at least 5 amino acids in length or comprise at least one CDR region, preferably a CDR3 region, more preferably a heavy chain CDR3 region.

In embodiments where the binding proteins of the invention comprise a fragment of any of the defined sequences (for example comprise a fragment of SEQ ID No. 2), e.g. are binding proteins comprising V_{H} and/or V_{L} domains of the invention, or are binding proteins comprising one or more CDRs of the invention, then these regions/domains are generally separated within the binding protein so that each region/domain can perform its biological function and so that the contribution to antigen binding is retained. Thus, the V_{H} and V_{L} domains may be separated by appropriate scaffold sequences/linker sequences and the CDRs may be separated by appropriate framework regions such as those found in naturally occurring antibodies. Thus, the V_{H}, V_{L} and individual CDR sequences of the invention can be provided within or incorporated into an appropriate framework or scaffold to enable antigen binding. Such framework sequences or regions can correspond to naturally occurring framework regions, FR1, FR2, FR3 and/or FR4, as appropriate to form an appropriate scaffold, or can correspond to consensus framework regions, for example identified by comparing various naturally occurring framework regions. Alternatively, non-antibody scaffolds or frameworks, e.g. T cell receptor frameworks can be used.

Appropriate sequences which can be used for framework regions are well known and documented in the art and any of these may be used. Preferred sequences for framework regions are one or more of the framework regions making up the V_{H} and/or V_{L} domains of the invention, i.e. one or more of the framework regions disclosed in SEQ ID No. 2 or in Table 2, or framework regions substantially homologous thereto, and in particular framework regions which allow the maintenance of antigen specificity, for example framework regions which result in substantially the same or the same 3D structure of the binding protein. In preferred embodiments, all four FR regions of SEQ ID NO: 2 (also shown in Table 2), or FR regions substantially homologous thereto, are found in the binding proteins of the invention.

In addition, although preferred binding proteins of the invention are made up of V_{H}, V_{L} or CDRs of the invention, it should be noted that the binding proteins of the invention also encompass one or more V_{H}, V_{L} or CDRs of the invention in combination with other V_{H}, V_{L} or CDRs not of the invention, provided that the tumor specific properties, and preferably the CD166 binding properties, of the binding proteins of the invention as outlined above are still present.

The term “heavy chain complementarity determining region” as used herein refers to regions of hypervariability within the heavy chain variable region (V_{H} domain) of an antibody molecule. The heavy chain variable region has three complementarity determining regions termed heavy chain complementarity determining region 1, heavy chain complementarity determining region 2 and heavy chain complementarity determining region 3 from the amino terminus to carboxy terminus. The heavy chain variable region also has four framework regions (FR1, FR2, FR3 and FR4 from the amino terminus to carboxy terminus). These regions separate the CDRs.

The term “heavy chain variable region” (V_{H} domain) as used herein refers to the variable region of a heavy chain of an antibody molecule.

The term “light chain complementarity determining region” as used herein refers to regions of hypervariability within the light chain variable region (V_{L} domain) of an antibody molecule. Light chain variable regions have three complementarity determining regions termed light chain complementarity determining region 1, light chain complementarity determining region 2 and light chain complementarity determining region 3 from the amino terminus to the carboxy terminus. The light chain variable region also has four framework regions (FR1, FR2, FR3 and FR4 from the amino terminus to carboxy terminus). These regions separate the CDRs.

The term “light chain variable region” (V_{L} domain) as used herein refers to the variable region of a light chain of an antibody molecule.

It should be noted that the Kabat nomenclature is followed herein, where necessary, in order to define the positioning of the CDRs (Kabat, E. A., Wu, T. T., Perry, H. M., Gottesman, K. S., and Foeller, C. (1991) Sequences of Proteins of Immunological Interest, 5th Ed, Public Health Service, National Institutes of Health, Bethesda, Md.).

Nucleic acid molecules comprising sequences encoding the binding proteins of the invention as defined above, or nucleic acid molecules substantially homologous thereto, form a yet further aspect of the invention. Preferred nucleic acid molecules are as defined in SEQ ID NO: 1, or nucleic acid molecules substantially homologous thereto. Other preferred nucleic acid molecules comprise sequences which encode the amino acid sequence of SEQ ID NO:2 or nucleic acid molecules substantially homologous thereto.

Fragments of the binding proteins of the invention as defined above, or sequences substantially homologous thereto, form a yet further aspect of the invention.

Accordingly, the invention provides a polypeptide comprising or consisting of a V_{L} domain of the invention as defined above, or a sequence substantially homologous thereto, or a polypeptide comprising or consisting of a V_{H} domain of the invention as defined above, or a sequence substantially homologous thereto.

Accordingly, the invention further provides a polypeptide comprising or consisting of one or more of the CDR regions of the invention as defined above, or sequences substantially homologous thereto.

When more than one CDR region is present, preferred combinations are also as described above.

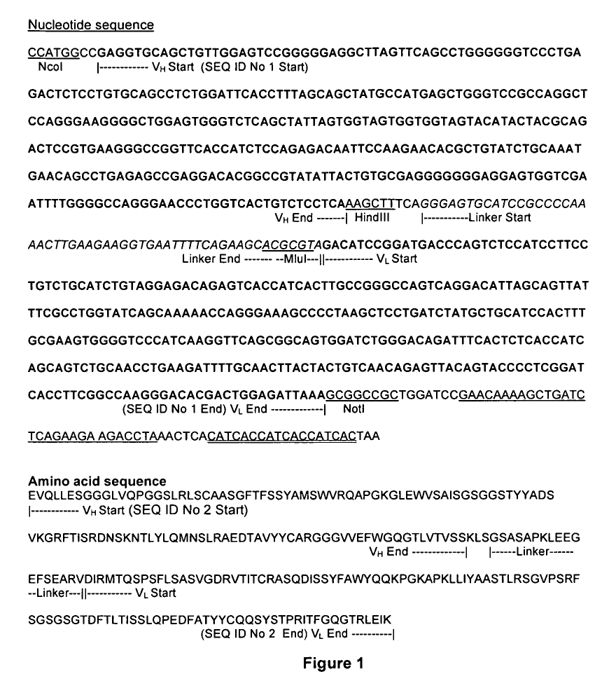

Nucleic acid molecules comprising sequences encoding such fragments of the binding proteins of the invention, or nucleic acid molecules substantially homologous thereto, form a yet further aspect of the invention. Preferred nucleic acid sequences encoding such fragments (e.g. V_{H} domains, V_{L} domains, and individual CDRs) can be found in SEQ ID NO: 1. Thus, a preferred nucleic acid molecule comprises SEQ ID NO:14, which encodes the V_{H} domain as shown in FIG. 1, or a nucleic acid molecule substantially homologous thereto and/or SEQ ID NO:15, which encodes the Vₗ domain as shown in FIG. 1, or a nucleic acid molecule substantially homologous thereto.

Other preferred nucleic acid molecules are those encoding a V_{H} or V_{L} domain of a binding protein of the present invention, e.g. those encoding SEQ ID NO:9 or SEQ ID NO:10, or sequences substantially homologous thereto.

The term “substantially homologous” as used herein in connection with an amino acid or nucleic acid sequence includes sequences having at least 50%, preferably at least 60%, more preferably at least 70%, most preferably at least 80%, and even more preferably at least 90%, 95%, 96%, 97%, 98% or 99%, sequence identity to the amino acid or nucleic acid sequence disclosed. Substantially homologous sequences of the invention thus include single or multiple base or amino acid alterations (additions, substitutions, insertions or deletions) to the sequences of the invention. At the amino acid level preferred substantially homologous sequences contain only 1, 2, 3, 4 or 5, preferably 1, 2 or 3, more preferably 1 or 2, altered amino acids, in one or more of the framework regions and/or one or more of the CDRs making up the sequences of the invention. Preferably said alterations are conservative amino acid substitutions.

The substantially homologous nucleic acid sequences also include nucleotide sequences that hybridize to the nucleic acid sequences disclosed (or their complementary sequences), e.g. hybridize to nucleotide sequences encoding one or more of the light chain or heavy chain CDRs of the invention, the light or heavy chain variable regions of the invention, or the binding proteins of the invention (or hybridize to their complementary sequences), under at least moderately stringent hybridization conditions.

The term “substantially homologous” also includes modifications or chemical equivalents of the amino acid and nucleotide sequences of the present invention that perform substantially the same function as the proteins or nucleic acid molecules of the invention in substantially the same way. For example any substantially homologous binding protein (or the substantially homologous nucleic acid encoding it) should retain the ability to specifically bind to cancer cells, preferably breast cancer cells. Preferably, any substantially homologous binding protein should retain the ability to specifically bind to the same antigen, i.e the CD166 antigen, and preferably to the same epitope thereof as recognized by the binding protein in question, for example, the same epitope or antigen recognised by the CDR domains of the invention or the V_{H} and V_{L} domains of the invention as described herein. Binding to the same epitope/antigen can be readily tested by methods well known and described in the art, e.g. using binding assays, e.g. a competition assay. Thus, a person skilled in the art will appreciate that binding assays can be used to find other antibodies and antibody fragments with the same binding specificities as the antibodies and antibody fragments of the invention. As exemplified, below, a competition binding assay can be used to find such other antibodies. The method described below is only one example of a suitable competition assay. The skilled person will be aware of other suitable methods and variations.

Before a competition assay is performed using flow cytometry, the minimal concentration of antibody of the invention (Ab1) that gives maximal binding against a fixed number of tumor cells, e.g. breast cancer cells, is determined. A total of 10⁶ cells are harvested from exponentially growing cultures and incubated with various antibody concentrations for 1 hr at 4° C. The cells are washed and incubated with a suitable detection antibody for an additional hour at 4° C. After washing, the cells are analyzed by flow cytometry. For each test antibody, a saturation curve is generated from the data by plotting median fluorescence against the antibody concentration.

For the competition assay, tumor cells, e.g. breast cancer cells, are prepared as above and treated in duplicate with a fixed concentration of antibody (Ab1). The fixed concentration is the minimal concentration of antibody that generates maximal binding against a fixed number of tumor cells as determined above. Immediately following the addition of the Ab1, varying concentrations of the potential inhibitory antibody (Ab2) is added to each tube and the mixture incubated for 1 hr at 4° C. Both the percent inhibition and change over maximum median fluorescence are calculated by subtracting the background fluorescence (PBS-5% FCS) from the median fluorescence reading for each test sample (Ab1+Ab2). The result is then divided by the median fluorescence of Ab1 alone (maximal binding) minus the background (see below). The percent of inhibition result is obtained by multiplying by 100. The mean of the replicates along with their respective standard error is plotted against antibody concentration. The following formula is used to calculate the percent inhibition:

PI=[(MF_{(Ab1+Ab2)}−MF_{Bgd})/(MF_{Ab1}−MF_{Bgd})]×100

where PI=percent inhibition; MF_{(Ab1+Ab2)}=median fluorescence measured for Ab1+Ab2 mixture; and MF_{Bgd}=background median fluorescence with PBS-5% FCS.

Accordingly, the invention provides a binding protein capable of binding an antigen on a tumor cell, preferably a breast cancer cell, wherein the binding protein can be identified by a method comprising:
(1) incubating a fixed number of tumor cells, preferably breast cancer cells, with a minimal concentration of a binding protein of the invention, preferably an antibody or antibody fragment (Ab1) that generates maximal binding against the fixed number of tumor cells and measuring median fluorescence of Ab1 (MF_{Ab1});
(2) testing two or more concentrations of a test binding protein (Ab2) by adding Ab2 to the Ab1 and tumor cells, and measuring median fluorescence (MF_{(Ab1+Ab2)});
(3) measuring background median fluorescence (MF_{bgd});
(4) calculating PI, wherein

PI=[(MF_{(Ab1+Ab2)}−MF_{Bgd})/(MF_{Ab1}−MF_{Bgd})]×100; and
(5) comparing the PI to a control PI value;

wherein, a PI that has a statistically significant difference from the control PI indicates that the test binding protein is capable of binding the antigen on the tumor cell. Preferably the statistically significant difference has a probability value of <0.05. Appropriate methods of determining statistical significance are well known and documented in the art and any of these may be used.

Any substantially homologous binding protein should also preferably retain the tumor specificity as described elsewhere herein, e.g. retain the ability to bind to tumor tissue without significantly binding to normal tissue. More preferably, it should retain the ability to bind specifically to CD166.

Substantially homologous sequences of proteins of the invention include, without limitation, conservative amino acid substitutions, or for example alterations which do not effect the VH, VL or CDR domains of the binding proteins, e.g. include scFv antibodies where a different linker sequence is used or binding proteins where tag sequences or other components are added which do not contribute to the binding of antigen, or alterations to convert one type or format of antibody molecule or fragment to another type or format of antibody molecule or fragment (e.g. conversion from Fab to scFv or vice versa), or the conversion of an antibody molecule to a particular class or subclass of antibody molecule (e.g. the conversion of an antibody molecule to IgG or a subclass thereof, e.g. IgG1 or IgG3).

A “conservative amino acid substitution”, as used herein, is one in which the amino acid residue is replaced with another amino acid residue having a similar side chain. Families of amino acid residues having similar side chains have been defined in the art, including basic side chains (e.g., lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Homology may be assessed by any convenient method. However, for determining the degree of homology between sequences, computer programs that make multiple alignments of sequences are useful, for instance Clustal W (Thompson, J. D., D. G. Higgins, et al. (1994). “CLUSTAL W: Improving the sensitivity of progressive multiple sequence alignment through sequence weighting, position-specific gap penalties and weight matrix choice”. Nucleic Acids Res 22: 4673-4680). If desired, the Clustal W algorithm can be used together with BLOSUM 62 scoring matrix (Henikoff S, and Henikoff J. G., 1992, Proc. Natl. Acad. Sci. USA 89: 10915-10919) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Programs that compare and align pairs of sequences, like ALIGN (E. Myers and W. Miller, “Optical Alignments in Linear Space”, CABIOS (1988) 4: 11-17), FASTA (W. R. Pearson and D. J. Lipman (1988), “Improved tools for biological sequence analysis”, PNAS 85:2444-2448, and W. R. Pearson (1990) “Rapid and sensitive sequence comparison with FASTP and FASTA” Methods in Enzymology 183:63-98) and gapped BLAST (Altschul, S. F., T. L. Madden, et al. (1997). “Gapped BLAST and PSI-BLAST: a new generation of protein database search programs”. Nucleic Acids Res. 25: 3389-3402), BLASTP, BLASTN, or GCG (Devereux et al., Nucleic Acids Res., 1984, 12: 387) are also useful for this purpose. Furthermore, the Dali server at the European Bioinformatics institute offers structure-based alignments of protein sequences (Holm, J. of Mol. Biology, 1993, Vol. 233: 123-38; Holm, Trends in Biochemical Sciences, 1995, Vol 20: 478-480; Holm, Nucleic Acid Research, 1998, Vol. 26: 316-9).

By way of providing a reference point, sequences according to the present invention having 50%, 60%, 70%, 80%, 90%, 95% homology etc. may be determined using the ALIGN program with default parameters (for instance available on Internet at the GENESTREAM network server, IGH, Montpellier, France).

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10[Na+])+0.41 (% (G+C)−600/l), or similar equation). Accordingly, the parameters in the wash conditions that determine hybrid stability are sodium ion concentration and temperature. In order to identify molecules that are similar, but not identical, to a known nucleic acid molecule, a 1% mismatch may be assumed to result in about a 1° C. decrease in Tm. For example, if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C.

By way of further example, sequences which “hybridize” are those sequences binding (hybridising) under non-stringent conditions (e.g. 6×SSC, 50% formamide at room temperature) and washed under conditions of low stringency (e.g. 2×SSC, room temperature, more preferably 2×SSC, 42° C.) or conditions of higher stringency (e.g. 2×SSC, 65° C.) (where SSC=0.15M NaCl, 0.015M sodium citrate, pH 7.2).

It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol. 3.

Generally speaking, sequences which hybridise under conditions of high stringency are preferred, as are sequences which, but for the degeneracy of the code, would hybridise under high stringency conditions.

The polypeptide, binding protein and nucleic acid molecules of the invention are generally isolated or purified molecules insofar as they are not present in situ within a human or animal body or a tissue sample derived from a human or animal body. The sequences may, however, correspond to or be substantially homologous to sequences as found in a human or animal body.

Thus, the term “isolated” or “purified” as used herein in reference to nucleic acid molecules or sequences and proteins or polypeptides, refers to such molecules when isolated from or purified from or substantially free of their natural environment, e.g. isolated from the human or animal body (if indeed they occur naturally), or refers to such molecules when produced by a technical process, i.e. includes recombinant and synthetically produced molecules.

Thus, when used in connection with a nucleic acid molecule, such a term may refer to a nucleic acid substantially free of material with which it is naturally associated such as other nucleic acids/genes or polypeptides. This term may also refer to a nucleic acid substantially free of cellular material or culture medium when produced by recombinant DNA techniques, or substantially free of chemical precursors, or other chemicals when chemically synthesized. An isolated or purified nucleic acid may also be substantially free of sequences which naturally flank the nucleic acid (i.e. sequences located at the 5′ and 3′ ends of the nucleic acid) from which the nucleic acid is derived or sequences which have been made to flank the nucleic acid (e.g. tag sequences or other sequence which have no therapeutic value) by for example genetic engineering.

Thus, when used in connection with a protein or polypeptide molecule such as light chain complementarity regions 1, 2 and 3, heavy chain complementarity regions 1, 2 and 3, light chain variable regions, heavy chain variable regions, and binding proteins of the invention, the term “isolated” or “purified” may refer to a protein substantially free of cellular material or other proteins from the source from which it is derived. In some embodiments, particularly where the protein is to be administered to humans or animals, such isolated or purified proteins are substantially free of culture medium when produced by recombinant techniques, or chemical precursors or other chemicals when chemically synthesized. Such isolated or purified proteins may also be free of flanking sequences such as those described above for the isolated nucleic acid molecules.

The term “nucleic acid sequence” or “nucleic acid molecule” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid molecules may be double stranded or single stranded. The nucleic acid molecules may be wholly or partially synthetic or recombinant.

A person skilled in the art will appreciate that the proteins and polypeptides of the invention, such as the light and heavy complementarity determining regions, the light and heavy chain variable regions, binding proteins, antibodies and antibody fragments, and immunoconjugates, may be prepared in any of several ways well known and described in the art, but are most preferably prepared using recombinant methods.

Accordingly, the nucleic acid molecules of the present invention may be cloned or synthesised by any appropriate method and may be incorporated in a known manner into an appropriate expression vector which ensures good expression of the proteins of the invention. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner which allows expression of the nucleic acid.

The invention therefore contemplates a recombinant expression vector containing a nucleic acid molecule of the invention, or a fragment thereof, and the necessary regulatory sequences for the transcription and translation of the protein sequence encoded by the nucleic acid molecule of the invention.

Suitable regulatory sequences may be derived from a variety of sources, including bacterial, fungal, viral, mammalian, or insect genes (For example, see the regulatory sequences described in Goeddel, Gene Expression Technology Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the invention may also contain a selectable marker gene which facilitates the selection of host cells transformed or transfected with a recombinant molecule of the invention. Examples of selectable marker genes are genes encoding a protein such as neomycin and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification (for example appropriate “tags” to enable purification and/or identification may be present, e.g. His tags or myc tags). For example, a proteolytic cleavage site may be added to the target recombinant protein to allow separation of the recombinant protein from the fusion moiety subsequent to purification of the fusion protein. Typical fusion expression vectors include PGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. The term “transformed host cell” as used herein is intended to also include cells capable of glycosylation that have been transformed with a recombinant expression vector of the invention. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of eukaryotic host cells and prokaryotic cells. For example, the proteins of the invention may be expressed in yeast cells or mammalian cells. Other suitable host cells can be found in Goeddel, Gene Expression Technology: Methods in Enzymology 185, Academic Press, San Diego, Calif. (1991). In addition, the proteins of the invention may be expressed in prokaryotic cells, such as Escherichia coli (Zhang et al., Science 303(5656): 371-3 (2004)).

Yeast and fungi host cells suitable for carrying out the present invention include, but are not limited to Saccharomyces cerevisiae, the genera Pichia or Kluyveromyces and various species of the genus Aspergillus. Examples of vectors for expression in yeast S. cerevisiae include pYepSec1 (Baldari. et al., Embo J. 6:229-234 (1987)), pMFa (Kurjan and Herskowitz, Cell 30:933-943 (1982)), pJRY 88 (Schultz et al., Gene 54:113-123 (1987)), and pYES2 (Invitrogen Corporation, San Diego, Calif.). Protocols for the transformation of yeast and fungi are well known to those of ordinary skill in the art (see Hinnen et al., Proc. Natl. Acad. Sci. USA 75:1929 (1978); Itoh et al., J. Bacteriology 153:163 (1983), and Cullen et al. (Bio/Technology 5:369 (1987)).

Mammalian cells suitable for carrying out the present invention include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No.1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the proteins of the invention may be expressed from plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, PAPS2022, PAPS2023, and PAPS2034)

Insect cells suitable for carrying out the present invention include cells and cell lines from Bombyx, Trichoplusia or Spodotera species. Baculovirus vectors available for expression of proteins in cultured insect cells (SF 9 cells) include the pAc series (Smith et al., Mol. Cell. Biol. 3:2156-2165 (1983)) and the pVL series (Lucklow, V. A., and Summers, M. D., Virology 170:31-39 (1989)). Some baculovirus-insect cell expression systems suitable for expression of the recombinant proteins of the invention are described in PCT/US/02442.

Alternatively, the proteins of the invention may also be expressed in non-human transgenic animals such as, rats, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866).

The proteins of the invention may also be prepared by chemical synthesis using techniques well known in the chemistry of proteins such as solid phase synthesis (Merrifield, J. Am. Chem. Assoc. 85:2149-2154 (1964); Frische et al., J. Pept. Sci. 2(4): 212-22 (1996)) or synthesis in homogenous solution (Houbenweyl, Methods of Organic Chemistry, ed. E. Wansch, Vol. 15 I and II, Thieme, Stuttgart (1987)).

N-terminal or C-terminal fusion proteins comprising the proteins of the invention conjugated with other molecules, such as proteins may be prepared by fusing, through recombinant techniques. The resultant fusion proteins contain a protein of the invention fused to the selected protein or marker protein, or tag protein as described herein. The proteins of the invention may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate. Examples of proteins which may be used to prepare fusion proteins or conjugates include cell binding proteins such as immunoglobulins, hormones, growth factors, lectins, insulin, low density lipoprotein, glucagon, endorphins, transferrin, bombesin, asialoglycoprotein glutathione-S-transferase (GST), hemagglutinin (HA), and truncated myc.

Accordingly, the invention provides a recombinant expression vector comprising one or more of the nucleic acid sequences of the invention or one or more of the nucleic acid sequences that encode the proteins of the invention (such as the light and heavy chain complementarity determining regions, the light and heavy chain variable regions, or the binding proteins, such as antibodies and antibody fragments).

Further, the invention provides a host cell comprising one or more of the recombinant expression vectors or one or more of the nucleic acid sequences of the invention, or a host cell expressing one or more of the proteins of the invention (such as the light and heavy chain complementarity determining regions, the light and heavy chain variable regions, or the binding proteins, such as antibodies and antibody fragments).

A yet further aspect of the invention provides a method of producing a protein of the present invention comprising a step of culturing the host cells of the invention. Preferred methods comprise the steps of (i) culturing a host cell comprising one or more of the recombinant expression vectors or one or more of the nucleic acid sequences of the invention under conditions suitable for the expression of the protein; and optionally (ii) isolating the protein from the host cell or from the growth medium/supernatant. Such methods of production may also comprise a step of purification of the protein product and/or formulating the product into a composition including at least one additional component, such as a pharmaceutically acceptable carrier or excipient.

In embodiments when the protein of the invention is made up of more than one polypeptide chain (e.g. certain fragments such as Fab fragments), then all the polypeptides are preferably expressed in the host cell, either from the same or a different expression vector, so that the complete proteins, e.g. binding proteins of the invention, can assemble in the host cell and be isolated therefrom.

The binding proteins of the invention have specificity for tumor cells, in particular breast carcinoma cells. Thus, the binding proteins of the invention can be used to detect or diagnose tumor cells, preferably breast carcinoma cells in vivo or in vitro. Thus, the binding proteins of the invention can target the body sites at which tumor cells, preferably breast carcinoma cells are present, whereupon the binding protein can act at the target site (e.g. target tissue, organ or cells).

The binding proteins of the invention preferably have specificity for the CD166 antigen. Thus, the binding proteins of the invention can be used to detect CD166 in vivo or in vitro. Thus, the binding proteins of the invention can target the body sites which express the CD166 antigen, whereupon the binding protein can act at the target site (e.g. target tissue, organ or cells).

The binding proteins of the invention preferably have the ability to induce antibody-dependent cell cytotoxicity (ADCC) of tumor cells, preferably breast cancer, lung cancer and/or prostate cancer cells. A suitable in vitro test for ADCC is described in Example 5. Thus, the binding proteins of the invention may for example cause at least 1%, 3% or 5% killing of tumour cells in vitro, preferably breast cancer, lung cancer and/or prostate cancer cells, preferably at least about 10%, 15% or 20% killing of tumour cells in vitro, preferably breast cancer, lung cancer and/or prostate cancer cells. Such an effect demonstrates that the binding proteins may be used to recruit the patients' immune system to combat tumour cells (i.e. can potentially be useful therapeutically without the need for an additional active agent). This is clearly an advantageous property.

The binding proteins of the invention preferably have the ability to inhibit the growth of tumour cells, preferably breast cancer cells. Said inhibition might be demonstrated in vitro or in vivo. A suitable in vitro test for growth inhibition is described in Example 6, which involves measurement of the fluorescence of cells labelled with a fixed amount of a dye. Cell division causes a reduction in fluorescence as the dye is divided between daughter cells, and if cell division is inhibited, there is less of a reduction of fluorescence. The fluorescence of different test populations can thus be compared to assess the growth inhibitory effect of candidate binding proteins.

Preferably, the binding proteins inhibit cell growth by at least 5% or 10%, more preferably at least 15, 20 or 25%, when tested over a period of 7 days in vitro.

A suitable in vivo test for growth inhibition is described in Example 9.

The binding proteins of the invention preferably have the ability to induce apoptosis of tumor cells, preferably breast cancer cells, optionally in combination with an additional pro-apoptotic agent.

Preferably the above described abilities are observed at a measurable or significant level and more preferably a statistically significant level, when compared to appropriate control conditions. Appropriate significance levels are discussed elsewhere herein.

Furthermore, the binding proteins of the invention can be conjugated to other entities and used to target these other entities to body sites at which tumor cells, preferably breast carcinoma cells, and/or cells which express CD166, are present. (Where the binding protein is an antibody molecule then such conjugates are also referred to as immunoconjugates). Such other entities could be labels or other detectable moieties, in which case these conjugate molecules would be useful for in vivo or in vitro diagnosis or imaging of body sites, in particular body sites afflicted with cancer. Appropriate labels and detectable moieties are discussed elsewhere herein. Alternatively the binding proteins of the invention could be conjugated to biologically active molecules or medically relevant agents such as toxins, enzymes, drugs, pre drugs, pro drugs or other small molecule compounds, or nucleic acid molecules (e.g. antisense molecules), in which case these conjugate molecules would be useful for targeted therapy, for example by targeting the drug, toxin or enzyme, etc., to cells or body sites at which carcinoma cells are present. Such biologically active molecules or medically relevant agents may be in an active form or in a form which is to be activated, for example in the body. In particular, such molecules could be used for targeting cancer cells, for example breast cancer cells.

Binding protein conjugates are thus preferred binding proteins of the invention. Preferred binding proteins to be used in the conjugates are full length (whole) antibodies, F(ab′)₂, Fab or scFv.

Methods for conjugating such other entities to the binding proteins of the invention are well known and described in the art and an appropriate method can readily be selected depending on the nature of the binding protein and the other entity to be conjugated. Thus, the other entities can be conjugated to the binding proteins of the invention either directly or via an intermediate, e.g. an appropriate linker. The conjugation might for example be covalent or non-covalent (e.g. the other entities can be conjugated to the binding protein via the formation of a complex with the binding protein or more conveniently with an intermediate linking entity such as a chemical group or a peptide tag). Such binding as a complex is for example appropriate for many radioisotopes.

In such embodiments, the binding proteins (e.g. the antibody or antibody fragment), together with the conjugated entity, could be included or incorporated in an artificial membrane, forming e.g. an artificial particle such as a micelle, liposome or nanoparticle. These particles would be guided to a target body site by virtue of the binding protein and could then fuse with the cells at the target site (or be internalized—see below), thereby releasing the conjugated entity, e.g. the biologically active molecules or medically relevant agents, from the inside of the artificial particle into the target cell, e.g. a tumor cell. Again, methods of incorporating molecules into such artificial membranes are well known and described in the art.

Some binding proteins (e.g. antibodies or antibody fragments) are capable of being internalized into the cells to which they become bound. Thus, in one embodiment of this invention the binding proteins of the invention are capable of being internalized. This property is particularly advantageous for use in conjugates, e.g. conjugates as discussed above, in embodiments where the biologically active molecule or medically relevant agent should be internalized with the antibody molecules. In general, the internalization of a binding protein is dependent on the antigen and the rate with which said antigen is recycled between cell membrane and cytosol. Thus, providing the binding protein interacts with its antigen (CD166) with a sufficient affinity such that the binding protein does not dissociate from the antigen (CD166) before the antigen is internalized, then the binding protein will also be internalized. This is clearly advantageous for certain embodiments, although in other embodiments, binding proteins which only display low internalisation rates are advantageous.

The internalization rate may thus vary between different binding proteins of the invention and for some binding proteins it may essentially correspond to the basic rate at which antibodies that bind to cell-surface molecules are typically turned over by their target cells (e.g. about 10% of the binding protein is internalized within 2 hours), but for other binding proteins the internalization rate may be higher, e.g. at least 10, 20, or 30% internalization within 30 minutes. The skilled person will be aware of suitable ways to assay internalisation, for example using temperature-differential fluorescence labeling on flow cytometry or confocal microscopy. An example of a suitable assay is described in Example 7, in which a secondary antibody labelled with a pH-sensitive dye (CypHer5E), which is minimally fluorescent at a basic pH (as found outside of cells) and maximally fluorescent at an acidic pH (as found inside of cells), is used.

Thus, it can be seen that a yet further aspect of the invention provides the binding proteins (e.g. binding protein conjugates) or other proteins of the invention as defined herein for use in therapy, diagnosis or imaging.

In addition, the invention provides compositions comprising the binding proteins of the invention, such as antibodies and antibody fragments, with one or more pharmaceutically acceptable excipient, carrier, diluent, buffer or stabilizer.

Such compositions can be used in any of aspects of the invention described herein where a binding protein is used, e.g. can be used in any of the methods, uses or kits as described herein.

A yet further aspect of the invention provides the use of the binding proteins (e.g. binding protein conjugates) or other proteins of the invention as defined herein in the manufacture of a composition or medicament for use in therapy, imaging or diagnosis.

Methods of treatment of a subject comprising the administration of an effective amount of a binding protein (e.g. binding protein conjugate) or other protein of the invention as defined herein to a subject, or to a sample (e.g. a blood sample) removed from a subject and which is subsequently returned to the subject, provide yet further aspects of the invention.

The in vivo methods as described herein are generally carried out in a mammal. Any mammal may be treated, for example humans and any livestock, domestic or laboratory animal. Specific examples include mice, rats, pigs, cats, dogs, sheep, rabbits, cows and monkeys. Preferably, however, the mammal is a human.

The terms “therapy” or “treatment” as used herein include prophylactic therapy, which may result in the prevention of disease. The terms “therapy” and “treatment” include combating or cure of disease but also include the controlling, reduction or alleviation of disease or one or more of the symptoms associated therewith. The antibodies of the invention can also be used in prognostic applications, e.g. for diseases in which CD166 levels are altered. Thus, the antibodies of the present invention can be used to prognose cancer. For example, it is reported that CD166 is shed into the bloodstream in more aggressive cancers. Thus, detection of elevated levels of CD166 in the blood using antibodies of the present invention can be useful to prognose aggressive, for example metastatic, cancers.

An “effective amount” as used herein can refer to a therapeutically effective amount or a prophylactically effective amount depending on the nature of the treatment. A therapeutically effective amount can be considered to be an amount necessary (at appropriate dosages and administration regimes) to achieve the desired therapeutic result. A prophylactically effective amount can be considered to be an amount necessary (at appropriate dosages and administration regimes) to achieve the desired prophylactic result. As indicated below, the amounts are likely to vary depending on the weight, age and sex of the patient, the severity of the disease and the ability of the binding protein to elicit a desired response in the individual.

The compositions of the present invention can be formulated according to any of the conventional methods known in the art and widely described in the literature. Thus, the active ingredient (i.e. the binding protein) may be incorporated, optionally together with other active substances (examples of which are as described below), with one or more conventional pharmaceutically acceptable carriers, diluents and/or excipients, etc., appropriate for the particular use for a composition, to produce conventional preparations which are suitable or can be made suitable for administration. They may be formulated as liquids, as semi-solids or as solids, e.g. liquid solutions, dispersions, suspensions, tablets, pills, powders, sachets, cachets, elixirs, emulsions, syrups, ointments, liposomes, suppositories, and the like. The preferred form depends on the intended mode of administration and therapeutic application. Preferably the composition comprising the binding protein of the invention is prepared in a form of an injectable or infusible solution.

The preferred mode of administration is parenteral, e.g. intraperitoneal, intravenous, subcutaneous, intramuscular, intracavity or transdermal, although any other appropriate mode may be used, for example oral administration. Intravenous injection or infusion is especially preferred. Any appropriate site of administration may be used. For example they may be administered locally and directly at the site where action is required or may be attached or otherwise associated, e.g. conjugated, with entities which will facilitate the targeting to an appropriate location in the body. Any physiologically compatible carrier, excipient, diluent, buffer or stabilizer can be used in the compositions of the invention. Examples of suitable carriers, excipients, diluents, buffers and stabilizers include one or more of water, saline, phosphate buffered saline, dextrose, glycerol, ethanol and the like, as well as combinations thereof. In some cases isotonic agents, e.g. sugars, polyalcohols (e.g. mannitol, sorbitol), or sodium chloride may be included. The compositions may additionally include lubricating agents, wetting agents, emulsifying agents, suspending agents, preserving agents, sweetening agents, flavouring agents, and the like. The compositions of the invention may be formulated so as to provide quick, sustained or delayed release of the active ingredient after administration to the subject by employing procedures well known in the art. As described above, preferably the composition is in a form suitable for injection and suitable carriers may be present at any appropriate concentration, but exemplary concentrations are from 1% to 20% and preferably from 5% to 10%.

Therapeutic compositions typically must be sterile and stable under conditions of manufacture and storage. Appropriate ways of achieving such sterility and stability are well known and described in the art.

In addition to a binding protein of the invention, the composition may further comprise one or more other active ingredients such as other agents which are useful for treating cancers, in particular breast cancer or other agents which are useful for treating diseases with which CD166 is associated or in which CD166 activity is detrimental. Suitable additional active agents for inclusion in a composition that is to be used in the treatment of mammals will be known to a person skilled in the art and can be selected depending on the nature of the disease which is to be treated by the composition. Suitable additional agents include antibodies which bind to other targets, cytokines, antimetabolites, alkylating agents, purine analogs and related inhibitors, pyrimidine analogs, folic acid analogs, vinca alkaloids, epipodopyllotoxins, antibiotics, L-aspaginase, estrogens, antiestrogens, androgens, antiandrogens, adrenocorticosteroides, progestines, adrenocortical suppressant, gonadotropin releasing hormone analogs, interferons, topoisomerase inhibitor, anthracenedione substituted urea, methyl hydrazine derivatives, platinum coordination complexes or chemical agents, or drugs controlling side effects. For breast cancer treatment suitable additional agents might include Herceptin, Doxil (Doxorubicin), Avastin or Taxotere.

Suitable additional agents may be selected from apoptosis-inducing agents, referred to herein as “pro-apoptotic agents”. The pro-apoptotic agent may be any suitable agent which can induce apoptosis and it may for example be an alkylating agent, a cross-linking agent, an intercalating agent, a nucleotide analogue, an inhibitor of spindle formation, and/or an inhibitor of topoisomerase I and/or II.

The skilled person will be well aware of suitable apoptosis-inducing agents, but by way of example staurosporine is mentioned herein.

Advantageously, the binding proteins of the present invention can act in synergy with another therapeutic agent. By “synergy” is meant that the effect of the binding protein and other agent when administered in combination is greater than the additive effect of the binding protein and other agent when each is administered alone as a single agent. The synergy may be in terms of cell growth inhibition, induction of apoptosis, or cytotoxicity, or any combination of any of the aforementioned. Preferably it is in terms of the induction of apoptosis. A synergistic effect between a binding protein of the invention and staurosporine was demonstrated in Example 8.

A synergistic effect can result in an improved therapeutic outcome. In some embodiments, it may allow the use of lower amounts of the binding protein and/or of the other agent, for example an amount which is sub-therapeutic if the binding protein or other agent is used alone.

Alternatively viewed, the binding proteins can preferably sensitise cancer cells to drug-induced apopotosis.

One aspect of the present invention is therefore a combination therapy in which a binding protein of the invention is used in conjunction with an apoptosis-inducing agent. The binding protein and the apoptosis-inducing agent may be used simultaneously, sequentially or separately.

Thus, in a further aspect, there is provided a method of treating cancer, comprising administration of a binding protein of the invention and separate, simultaneous or sequential administration of a pro-apoptotic agent to a subject in need thereof.

Alternatively viewed, there is provided a binding protein as defined herein for use in combination with a pro-apoptotic agent in therapy, e.g. in the treatment of cancer.

Thus, there is provided the use of binding protein of the invention in the manufacture of a medicament for use in combination with a pro-apoptotic agent in the treatment of disease, e.g. cancer.

Thus, in one embodiment the medicament may further comprise a pro-apoptotic agent.

The medicament may be in the form of a single composition comprising both the binding protein of the invention and the pro-apoptotic agent, or it may be in the form of a kit or product containing them for separate administration.

In another aspect, the invention provides a product containing a binding protein of the invention together with a pro-apoptotic agent as a combined preparation for separate, simultaneous or sequential use in therapy, e.g. in the treatment of cancer.

As noted above, when the binding protein as defined herein is used in conjunction with a pro-apoptotic agent, then the two different agents may be present in the same pharmaceutical composition, or they may be administered separately. Separate administration may include administration at substantially the same time but via different routes of administration, or by administration at different locations. Separate administration may also include administration at different times, e.g. up to 1, 2, 3, 4, 5, 6 or 12 hours apart.

Suitable doses of the binding protein of the invention and the other active ingredients (if included) will vary from patient to patient and will also depend on the nature of the particular disease. Preferably, said dosages constitute a therapeutically effective amount or a prophylactically effective amount, depending on the nature of the treatment involved. Suitable doses can be determined by the person skilled in the art or the physician in accordance with the weight, age and sex of the patient and the severity of the disease. The ability of the binding protein to elicit a desired response in the individual will also be a factor. Exemplary daily doses are: 0.1 to 250 mg/kg, preferably 0.1 to 200 or 100 mg/kg, more preferably 1 to 50 or 1 to 10 mg/kg, of the active ingredient. This can be administered as a single unit dose or as multiple unit doses administered more than once a day. It is to be noted however that appropriate dosages may vary depending on the patient and that for any particular subject, specific dosage regimes should be adjusted over time according to the individual needs of the patient. Thus, the dosage ranges set forth herein are to be regarded as exemplary and are not intended to limit the scope or practice of the claimed composition.

Yet further aspects are methods of diagnosis or imaging of a subject comprising the administration of an appropriate amount of a binding protein (e.g. binding protein conjugate) or other protein of the invention as defined herein to the subject and detecting the presence and/or amount and/or the location of the binding protein or other protein of the invention in the subject.

Appropriate diseases to be treated, imaged or diagnosed in accordance with the above described uses and methods include any disease associated with molecules recognised by the proteins of the invention (e.g. any disease in which CD166 is associated or plays a role), in particular cancer (e.g. cancers which express or aberrantly express CD166), preferably breast cancer, or any other diseases in which CD166 is associated or plays a role, e.g. diseases associated with the presence or overexpression of CD166 or where inhibition of CD166 activity might be advantageous.

The binding proteins of the invention bind selectively to cancer cells, preferably breast cancer cells, and not significantly to normal cells. Therefore the binding proteins can be used in the diagnosis, imaging or therapy of cancer, in particular breast cancer. As stated above, the inventors have shown that the binding proteins of the invention bind to CD166. Thus, the specificity of the binding proteins for tumor antigens makes it useful in the diagnosis, imaging or therapy of cancer.

In one embodiment of the invention, cancer includes, without limitation, one or more of cervical cancer, uterine cancer, ovarian cancer, pancreatic cancer, kidney cancer, gallbladder cancer, liver cancer, head and neck cancer, squamous cell carcinoma, gastrointestinal cancer, breast cancer (such as carcinoma, ductal, lobular, and nipple), prostate cancer, testicular cancer, lung cancer, non-small cell lung cancer, non-Hodgkin's lymphoma, multiple myeloma, leukemia (such as acute lymphocytic leukemia, chronic lymphocytic leukemia, acute myelogenous leukemia, and chronic myelogenous leukemia), brain cancer (e.g. astrocytoma, glioblastoma, medulloblastoma), neuroblastoma, sarcomas, colon cancer, rectum cancer, stomach cancer, anal cancer, bladder cancer, pancreatic cancer, endometrial cancer, plasmacytoma, lymphomas, retinoblastoma, Wilm's tumor, Ewing sarcoma, melanoma and other skin cancers. Preferred cancers are those which show aberrant expression of CD166 including one or more of melanoma, prostate cancer, breast cancer, colorectal carcinoma, bladder cancer, ovarian cancer, pancreas cancer, lung cancer, brain cancer and esophageal squamous cell carcinoma. Preferred cancers are one or more of breast cancer, prostate cancer, lung cancer, ovarian cancer, colon cancer, kidney cancer and brain cancer (in particular glioblastoma). Especially preferred cancers are one or more of breast cancer, prostate cancer, lung cancer and kidney cancer. Especially preferred breast cancers are breast cancer carcinoma, ductal breast cancer, lobular breast cancer, and nipplebreast cancer, with breast carcinoma being most preferred.

Any reference herein to “cancer” or “tumor” should be understood to include a reference to any of the cancer types listed above, in particular breast cancer.

In a preferred embodiment, the binding proteins are antibodies or antibody fragments of the invention, the scFv or Fab or IgG form being especially preferred.

In addition, cancer cells may be evaluated to determine their susceptibility to the treatment methods of the invention by, for example, obtaining a sample of the cancer cells from a subject and determining the ability of the cancer cells in the sample to bind to the binding proteins of the invention, preferably antibodies or antibody fragments.

Accordingly, the present invention includes diagnostic methods, agents, and kits that can be used by themselves, or prior to, during or subsequent to the therapeutic method of the invention in order to determine whether or not cancer cells, preferably breast cancer cells, are present that express the antigen and can bind to the binding proteins of the invention, preferably antibodies and antibody fragments.

In one embodiment, the invention provides a method of diagnosing disease, preferably cancer, in a mammal comprising the step of:
(1) contacting a test sample taken from said mammal with any one or more of the binding proteins of the invention.

In a further embodiment, the invention provides a method of diagnosing disease, preferably cancer, in a mammal comprising the steps of:
(1) contacting a test sample taken from said mammal with one or more of the binding proteins of the invention;
(2) measuring the presence and/or amount and/or location of binding protein-antigen complex in the test sample; and, optionally
(3) comparing the presence and/or amount of binding protein-antigen complex in the test sample to a control.

In one embodiment, the antigen is CD166.

In the above methods, said contacting step is carried out under conditions that permit the formation of a binding protein-antigen complex. Appropriate conditions can readily be determined by a person skilled in the art.

In the above methods any appropriate test sample may be used, for example biopsy cells, tissues or organs suspected of being affected by cancer, histological sections or blood.

In the above methods the presence of an amount of binding protein-antigen complex in the test sample would be indicative of the presence of cancer cells. For a positive diagnosis to be made, generally the amount of binding protein-antigen complex in the test sample is greater than, preferably significantly greater than, the amount found in an appropriate control sample. More preferably, the significantly greater levels are statistically significant, preferably with a probability value of <0.05. Appropriate methods of determining statistical significance are well known and documented in the art and any of these may be used.

Appropriate control samples could be readily chosen by a person skilled in the art, for example, in the case of diagnosis of a particular disease, an appropriate control would be a sample from a subject that did not have that disease.

For use in the diagnostic or imaging applications, the binding proteins of the invention, preferably antibodies or antibody fragments, may be labeled with a detectable marker such as a radio-opaque or radioisotope, such as ³H, ¹⁴C, ³²P, ³⁵S, ¹²³I, ¹²⁵I, ¹³¹I; a radioactive emitter (e.g. α, β or γ emitters); a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion; or a chemical moiety such as biotin which may be detected by binding to a specific cognate detectable moiety, e.g. labelled avidin/streptavidin. As described above, methods of attaching a label to a binding protein, such as an antibody or antibody fragment, are known in the art. Such detectable markers allow the presence, amount or location of binding protein-antigen complexes in the test sample to be examined.

Another aspect of the invention is a method of diagnosing disease, preferably cancer, in a mammal comprising the steps of:

(1) measuring the presence and/or amount of antibodies of the invention in a test sample taken from said mammal; and optionally

(2) comparing the presence and/or amount of antibodies of the invention in the test sample to a control.

In one embodiment, the amount of antibodies of the invention is measured by measuring the amount of antibodies of the invention in the test sample, for example by ELISA, e.g. using CD166 as antigen. In another embodiment, the amount of antibodies of the invention is measured by measuring the expression levels of nucleic acids encoding the antibodies of the invention in the test sample, for example by RT-PCR.

The invention also includes diagnostic or imaging agents comprising the binding proteins of the invention (e.g. antibodies or antibody fragments) attached to a label that produces a detectable signal, directly or indirectly. Appropriate labels are described elsewhere herein.

The invention further includes kits comprising one or more of the binding proteins or compositions of the invention or one or more of the nucleic acid molecules encoding the binding proteins of the invention, or one or more recombinant expression vectors comprising the nucleic acid sequences of the invention, or one or more host cells comprising the recombinant expression vectors or nucleic acid sequences of the invention. Preferably said kits are for use in the methods and uses as described herein, e.g. the therapeutic, diagnostic or imaging methods as described herein, or are for use in the in vitro assays or methods as described herein. The binding protein in such kits may preferably be a binding protein conjugate as described elsewhere herein, e.g. may be conjugated to a detectable moiety. Preferably said kits comprise instructions for use of the kit components, for example in diagnosis. Preferably said kits are for diagnosing cancer and optionally comprise instructions for use of the kit components to diagnose cancer.

The invention further includes a kit for diagnosing cancer comprising one or more of the binding proteins of the invention and optionally instructions for the use thereof to diagnose the cancer. The invention also includes a kit for diagnosing cancer comprising a binding protein, preferably an antibody or antibody fragment as described herein, most preferably an antibody or antibody fragment that binds to CD166, and optionally instructions for the use thereof to diagnose cancer.

The binding proteins as defined herein may also be used as molecular tools for in vitro or in vivo applications and assays. As the binding proteins have an antigen binding site, these can function as members of specific binding pairs and these molecules can be used in any assay where the particular binding pair member is required. For example, in the embodiments when the binding proteins are antibodies or antibody fragments which can bind particular antigens such as CD166 these molecules can be used in any assay requiring an antibody with a specificity for that particular antigen, for example can be used in any assay where detection of CD166 is required or desired.

Thus, yet further aspects of the invention provide a reagent which comprises a binding protein as defined herein and the use of such binding proteins as molecular tools, for example in in vitro or in vivo assays, e.g. in in vitro or in vivo assays to detect CD166. Thus, a yet further aspect of the invention provides a method of binding CD166 comprising contacting a composition containing CD166 (or a molecule comprising CD166 or a fragment thereof) with the binding proteins of the invention or a conjugate thereof. The invention further provides a method of detecting CD166 (or a molecule comprising CD166 or a fragment thereof), comprising contacting a composition suspected of containing such a CD166 molecule with the binding proteins of the invention or a conjugate thereof, under conditions effective to allow the formation of CD166/binding protein complexes and detecting the complexes so formed.

CD166 has been linked with cell migration and development and with cancer progression and aberrant expression of CD166 has been implicated in several human tumors including melanoma, prostate cancer, breast cancer, colorectal carcinoma, bladder cancer, ovarian cancer, pancreas cancer, lung cancer, brain cancer and esophageal squamous cell carcinoma.

Regardless of the mechanism, the binding proteins of the invention could be used to modulate the signaling of CD166 involved in cell migration, cancer development, or cancer progression.

Accordingly, the invention includes the use of the binding proteins of the invention to modulate the activity of CD166. For example, the binding proteins of the invention can be used to interfere with or inhibit CD166 activity. The binding proteins of the invention may also be used to enhance CD166 activity.

As an example, the binding proteins may be used to induce apoptosis of cells, as exemplified in Example 8. The induction of apoptosis may be assayed using Annexin V as described in Example 8.

The binding proteins of the invention may also be used to produce further binding proteins which are specific for tumor cells, for example breast cancer cells or for CD166. Such uses involve for example the addition, deletion, substitution or insertion of one or more amino acids in the amino acid sequence of a parent binding protein to form a new binding protein, wherein said parent binding protein is one of the binding proteins of the invention as defined elsewhere herein, and testing the resulting new binding protein to identify binding proteins specific for tumor cells or for CD166. Such methods can be used to form multiple new binding proteins which can all be tested for their ability to bind tumor cells or to bind to CD166. Preferably said addition, deletion, substitution or insertion of one or more amino acids takes place in one or more of the CDR domains.

Such modification or mutation to a parent binding protein can be carried out in any appropriate manner using techniques well known and documented in the art, for example by carrying out methods of random or directed mutagenesis. If directed mutagenesis is to be used then one strategy to identify appropriate residues for mutagenesis utilizes the resolution of the crystal structure of the binding protein-antigen complex, e.g. the Ab-Ag complex, to identify the key residues involved in the antigen binding (Davies D. R., Cohen G. H. 1996. Interactions of protein antigens with antibodies. Proc Natl. Acad. Sci. U.S.A. 93, 7-12). Subsequently, those residues can be mutated to enhance the interaction. Alternatively, one or more amino acid residues can simply be targeted for directed mutagenesis and the effect on binding to tumor cells or CD166 assessed.

Random mutagenesis can be carried out in any appropriate way, e.g. by error-prone PCR, chain shuffling or mutator E. coli strains.

Thus, one or more of the V_{H} domains of the invention can be combined with a single V_{L} domain or a repertoire of V_{L} domains from any appropriate source and the resulting new binding proteins tested to identify binding proteins specific for tumor cells or CD166. Conversely, one or more of the V_{L} domains of the invention can be combined with a single V_{H} domain or repertoire of V_{H} domains from any appropriate source and the resulting new binding proteins tested to identify binding proteins specific for tumor cells or CD166.

Similarly, one or more, or preferably all three CDRs of the V_{H} and/or V_{L} domains of the invention can be grafted into a single V_{H} and/or V_{L} domain or a repertoire of V_{H} and/or V_{L} domains, as appropriate, and the resulting new binding proteins tested to identify binding proteins specific for tumor cells or CD166.

The targeted mutations of the CDRs, especially CDR3 of the light and/or heavy chains, have been shown to be an effective technique for increasing antibody affinity and are preferred. Preferably, blocks of 3 to 4 amino acids of the CDR3 or specific regions called “hot-spots” are targeted for mutagenesis.

“Hot spots” are the sequences where somatic hypermutation takes place in vivo (Neuberger M. S and Milstein C. 1995. Somatic hypermutation. Curr. Opin. Immunol. 7, 248-254). The hotspot sequences can be defined as consensus nucleotide sequences in certain codons. The consensus sequence is the tetranucleotide, RGYW, in which R can be either A or G, Y can be C or T and W can be either A or T (Neuberger M. S and Milstein C. 1995. Somatic hypermutation. Curr. Opin. Immunol. 7, 248-254). In addition, the serine residues encoded by the nucleotides AGY are predominantly present in the CDRs regions of the variable domain over those encoded by TCN corresponding to a potential hot-spot sequences (Wagner S. D., Milstein C. and Neuberger M. S. 1995. Codon bias targets mutation. Nature, 376, 732).

Thus, the nucleotide sequence of the CDRs of the heavy and light chains of each antibody of the invention can be scanned for the presence of the hot-spot sequences and AGY codons. The identified hot-spots of the CDR regions of the light and heavy chain can then optionally be compared to the germinal sequences of the heavy and light chains using the International ImMunoGen Tics database (IMGT, http://imgt.cines.fr/textes/vquest/) (Davies D. R., Padlan E. A. and Sheriff S. 1990. Antibody-antigen complexes. Annu. Rev. Biochem. 59, 439-473). A sequence, identical to the germ line, suggest that somatic mutation has not occurred; therefore random mutations can be introduced mimicking the somatic events occurring in vivo or alternatively, site directed mutagenesis can be carried out, e.g. at the hot spots and/or AGY codons. In contrast, a different sequence shows that some somatic mutations have already occurred. It will remain to be determined if the in vivo somatic mutation was optimal.

Preferred hot-spots for mutation are those that code for exposed amino acids and preferably those that encode amino acids which form part of the antigen binding sites. Other preferred hot-spots for mutation are those that code for non-conserved amino acids. The hot-spots that code for buried or conserved amino acids within the CDRs are preferably not mutagenized. These residues are usually critical for the overall structure and are unlikely to interact with the antigen since they are buried.

Methods of carrying out the above described manipulation of amino acids and protein domains are well known to a person skilled in the art. For example, said manipulations could conveniently be carried out by genetic engineering at the nucleic acid level wherein nucleic acid molecules encoding appropriate binding proteins and domains thereof are modified such that the amino acid sequence of the resulting expressed protein is in turn modified in the appropriate way.

Testing the ability of one or more new binding proteins to specifically bind to tumor cells or CD166 can be carried out by any appropriate method which are well known and described in the art. Tumor cells, e.g. breast cancer carcinoma cell lines are widely available (see the Examples) and these or CD166, which is commercially available e.g. from R&D systems, can readily be used to assay binding, for example by conventional methods such as ELISA, affinity chromatography, etc.

The new binding proteins produced by these methods will preferably have a higher or enhanced affinity (or at least an equivalent affinity) for tumor cells or CD166 as the parent binding protein and can be treated and used in the same way as the binding proteins of the invention as described elsewhere herein (e.g. for therapy, diagnosis, in compositions etc).

New binding proteins produced, obtained or obtainable by these methods form a yet further aspect of the invention.

Other features and advantages of the present invention will become apparent from the above detailed description. It should be understood, however, that the above detailed description and the following specific examples, while indicating preferred embodiments of the invention, are given by way of illustration only, since various changes and modifications within the spirit and scope of the invention will become apparent to those skilled in the art from the detailed description.

The invention will now be described in more detail in the following non-limited examples with reference to the Tables and Figures in which:

Table 1 lists some of the sequences disclosed herein.

Table 2 shows the framework regions of clone EJ212/007-Cl2-5 (scFv) (according to Kabat).

Table 3 shows the method of IHC scoring used for human tissue samples.

Table 4 shows IHC data for clone EJ212/007-Cl2-5 (scFv)

Table 5 shows IHC data for clone EJ212/007-Cl2-5 IgG on some human normal and corresponding tumor tissues.

Table 6 shows the list of Example 10 of peptides used for mass fingerprinting that matched with the CD166 protein.

FIG. 1 shows the nucleotide and amino acid sequence of inter alia the heavy and light chain of clone EJ212/007-Cl2-5 scFv. ScFv were cloned via NcoI/NotI site into pHOG21 (3.7 Kb). The restriction sites used for initial cloning (NcoI, HindIII, MluI and NotI) are italicized and underlined. The linker sequence between V_{H} and V_{L} is in italic. The c-myc epitope and 6 His are underlined and double underlined, respectively.

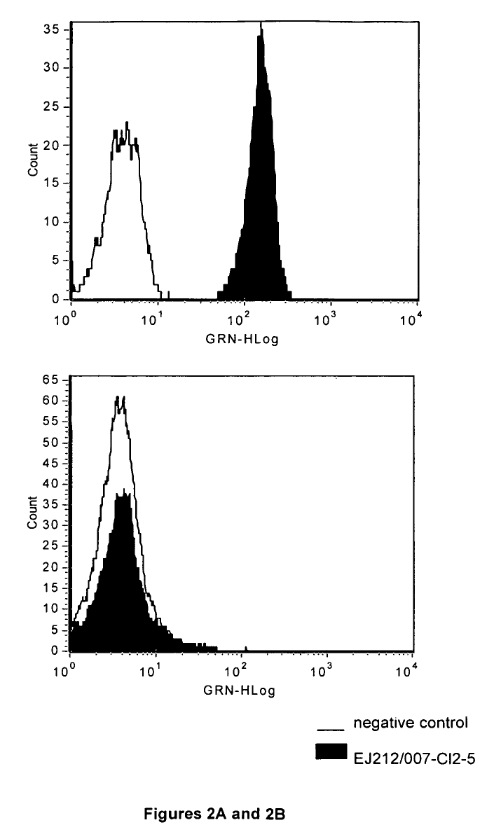

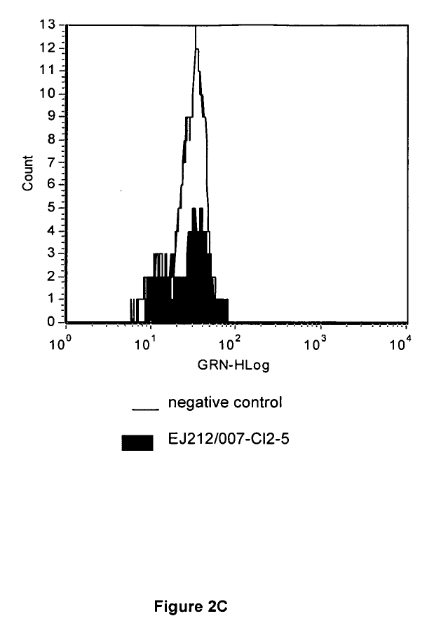

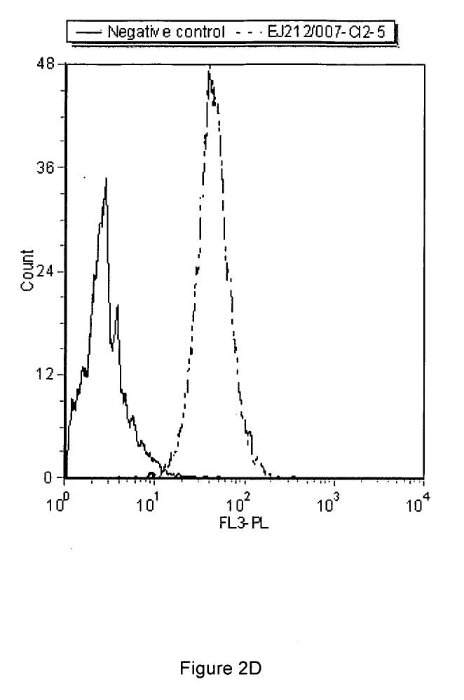

FIG. 2 shows the flow cytometry results of Example 2. FIG. 2 A shows binding of EJ212/007-Cl2-5 to MDA-MB 231 cells, FIG. 2 B shows that there is no or insignificant binding to PBL and FIG. 2 C shows that there is no or insignificant binding to granulocytes. FIG. 2D shows binding of EJ212/007-Cl2-5 to SW900 cells.

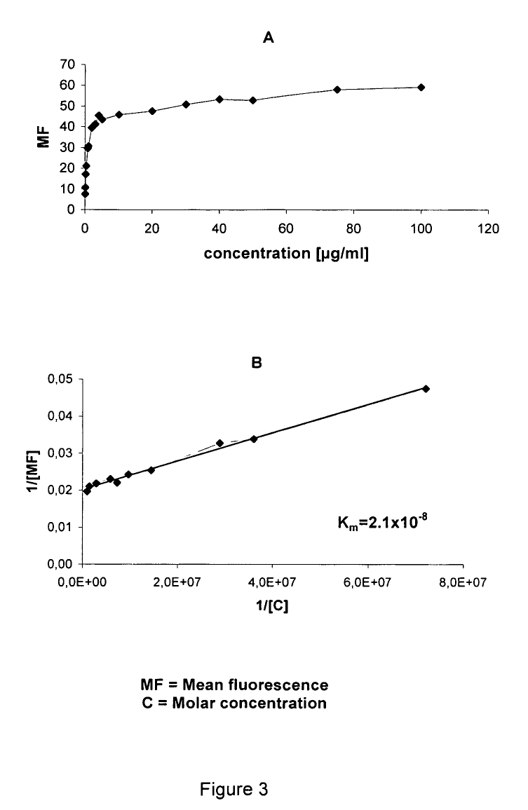

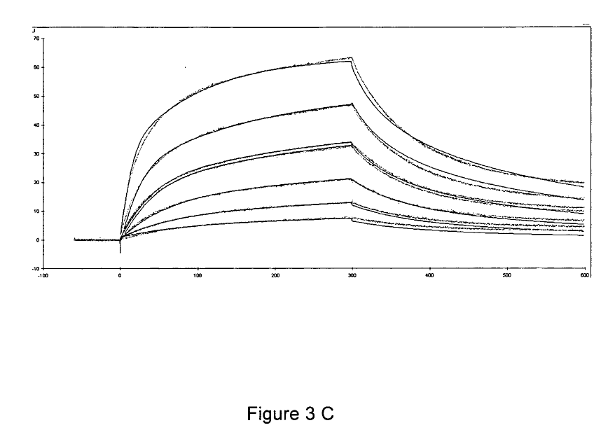

FIG. 3 shows the binding affinity results of Example 3, showing saturation curve of EJ212/007-Cl2-5 in FIG. 3 A and Lineweaver-Burk plot of EJ212/007-Cl2-5 in FIG. 3 B. FIG. 3 C shows the results of Biacore analysis of the binding affinity of EJ212/007-Cl2-5 Fab fragments. The binding curves are shown in FIG. 3 C and the K_{d} value was measured as 2.9×10⁻⁸ M for the EJ212/007-Cl2-5 Fab.

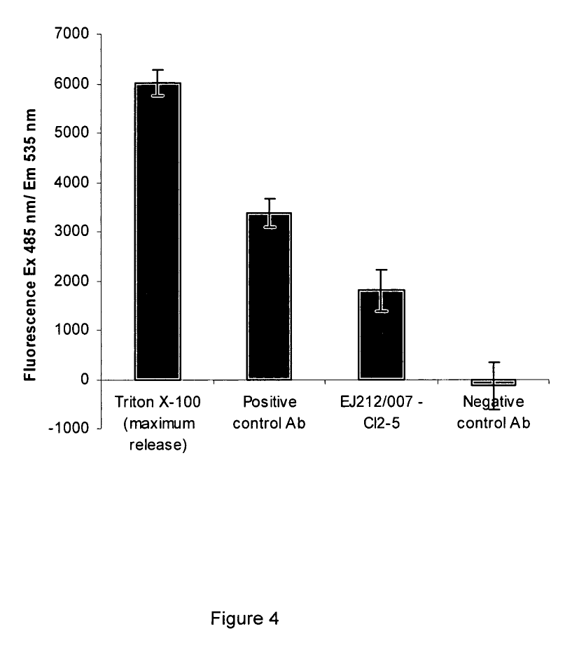

FIG. 4 shows antibody-dependent cell cytotoxicity (ADCC) results by EJ212/007-Cl2-5 IgG of Example 5. The figure shows that EJ212/007-Cl2-5 is able to induce ADCC of PM-1 cells, whereas the negative control antibody did not show a cytotoxic effect. Error bars represent the standard deviations calculated from quadruplicates used for each sample. Triton X-100 samples show the maximum levels of cytotoxicity.

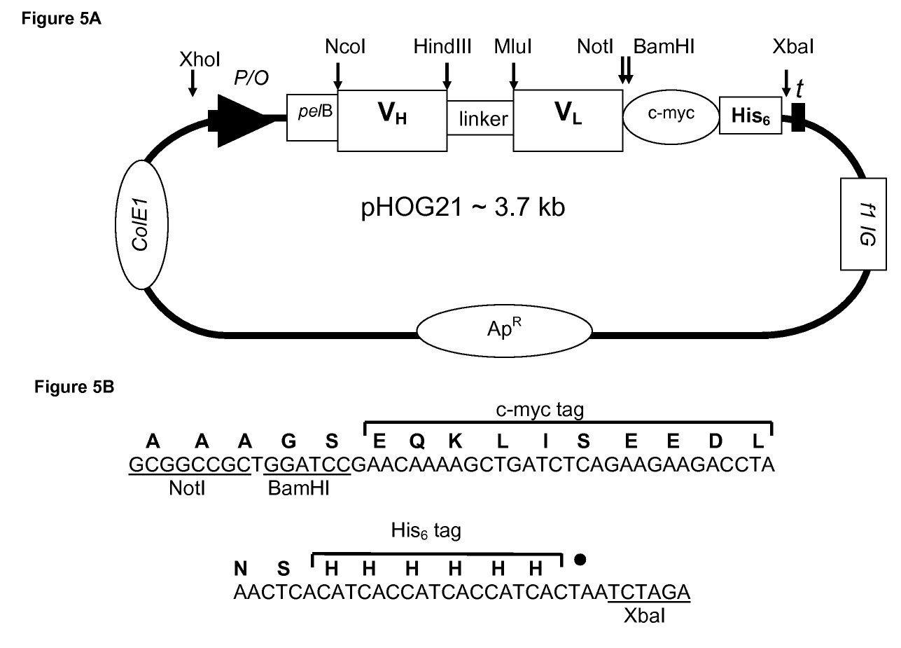

FIG. 5A shows the scFv expression vector pHOG21. ApR, Ampicillin resistance gene; ColE1, origin of DNA replication; fIIG, intergenic region of phage f1; c-myc, epitope recognized by the monoclonal antibody 9E10; His6, six histine residues; pelB, signal peptide of bacterial pectate lyase; P/O, wild type lac promoter operator. FIG. 5B shows the nucleotide and amino acids sequences of the C-terminal coding region.

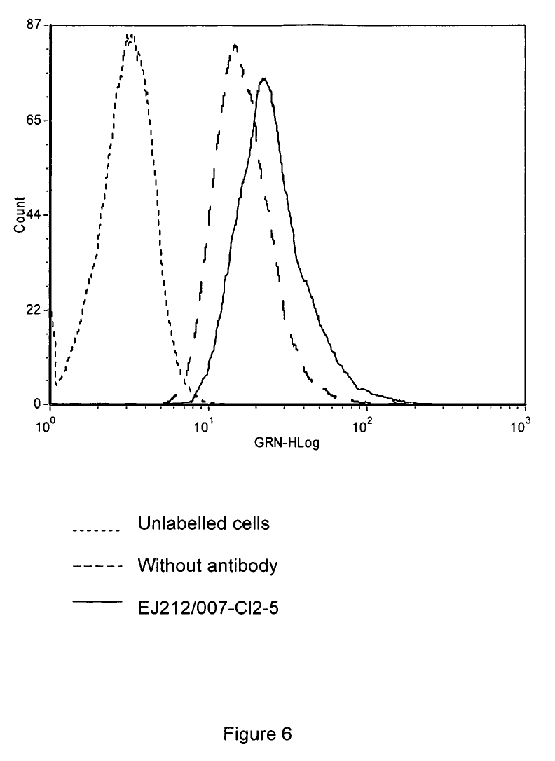

FIG. 6 shows the mean fluorescence intensity of CFSE-labeled cells of Example 6 after 7 days incubation in the presence of EJ212/007-Cl2-5 IgG compared to CFSE-labeled cells incubated without IgG. The higher the fluorescence intensity, the less cell division there has been, so a higher fluorescence intensity is indicative of cell cycle inhibition by the antibody

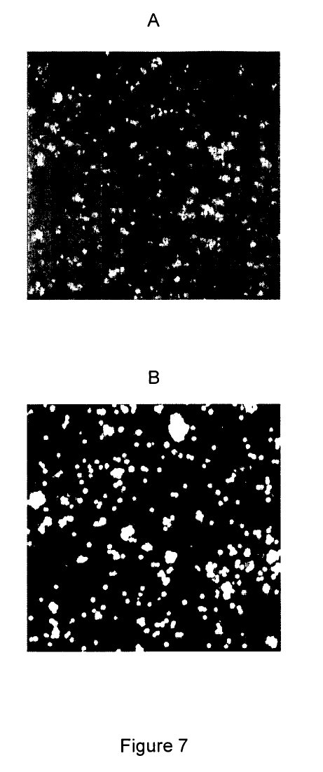

FIG. 7A shows the internalization of Example 7 of EJ212/007-Cl2-5 scFv in PM-1 cells after 30 minutes incubation at 37° C. FIG. 7B shows the positive control for internalization.

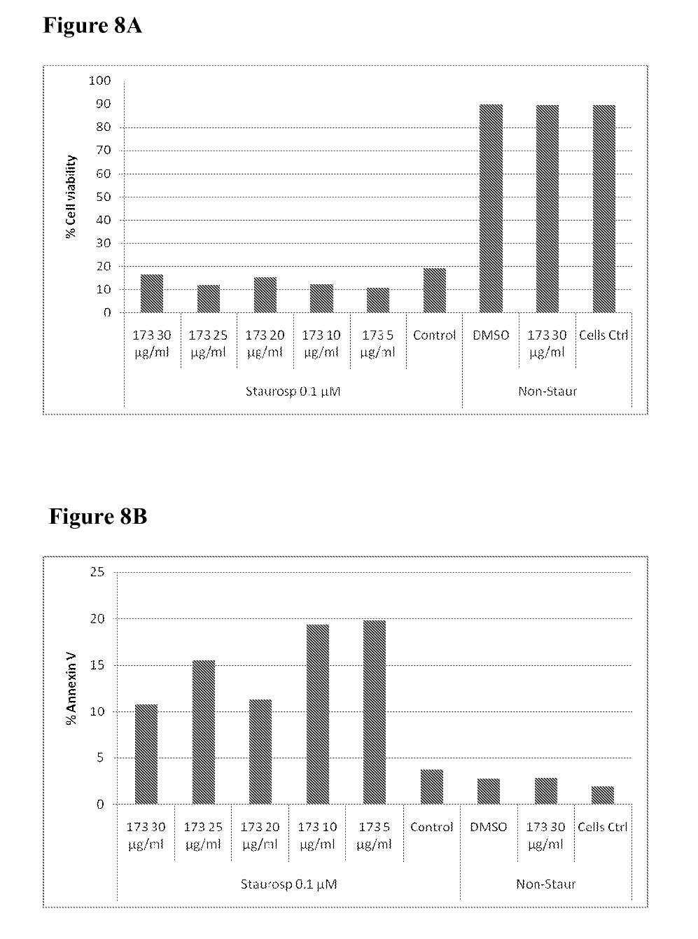

FIG. 8 A shows reduced cell viability after incubation of BT474 cells with EJ212/007-Cl2-5 IgG in presence of Staurosporine. FIG. 8 B shows the positive Annexin V staining of BT474 cells of Example 8 after incubation with EJ212/007-Cl2-5 IgG in presence of Staurosporine.

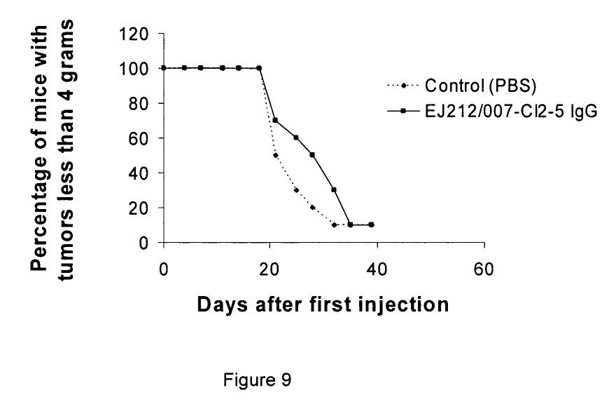

FIG. 9 shows the results of EJ212/007-Cl2-5 IgG treatment of MDA-MB 231-bearing mice of Example 9. The results are displayed as number of mice with tumors weighing less than 4 grams after injection of the antibody. The figure shows that the EJ212/007-Cl2-5 IgG reduced tumor growth compared the tumors from the control group that received PBS.

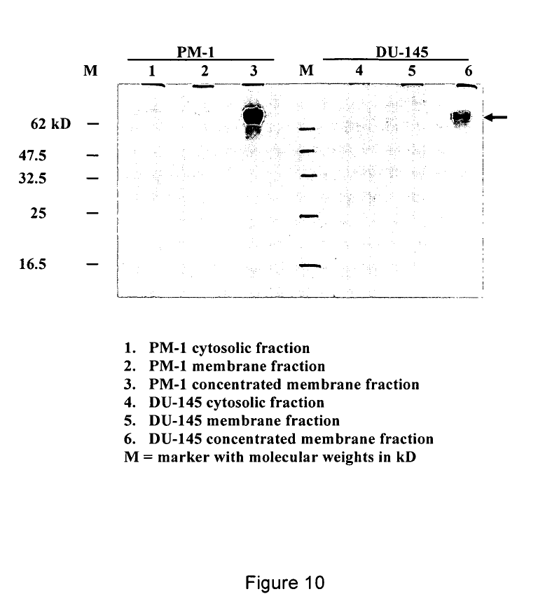

FIG. 10 shows the western blot analysis of Example 10 with EJ212/007-Cl2-5 IgG on the cytosolic and membrane fraction (before and after concentration) of PM-1 and DU-145 cells. A clear band for the potential EJ212/007-Cl2-5 antigen is seen in the purified membrane fraction after concentration, as indicated by the arrow.

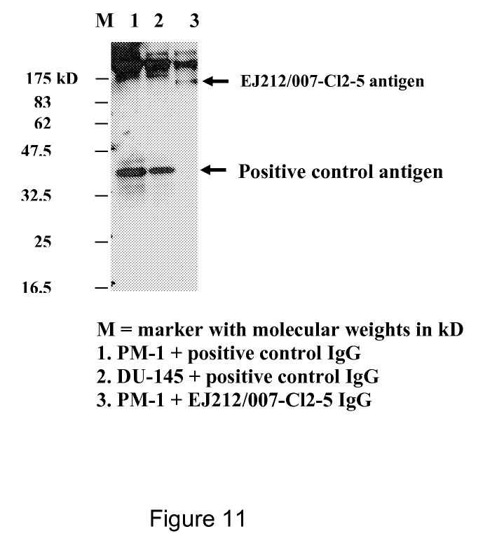

FIG. 11 shows the western blot analysis of Example 10 of the protein captured with EJ212/007-Cl2-5 IgG from PM-1 cells. The indicated band for EJ212/007-Cl2-5 antigen was excised and used for mass spectrometry analysis of the digested peptides.

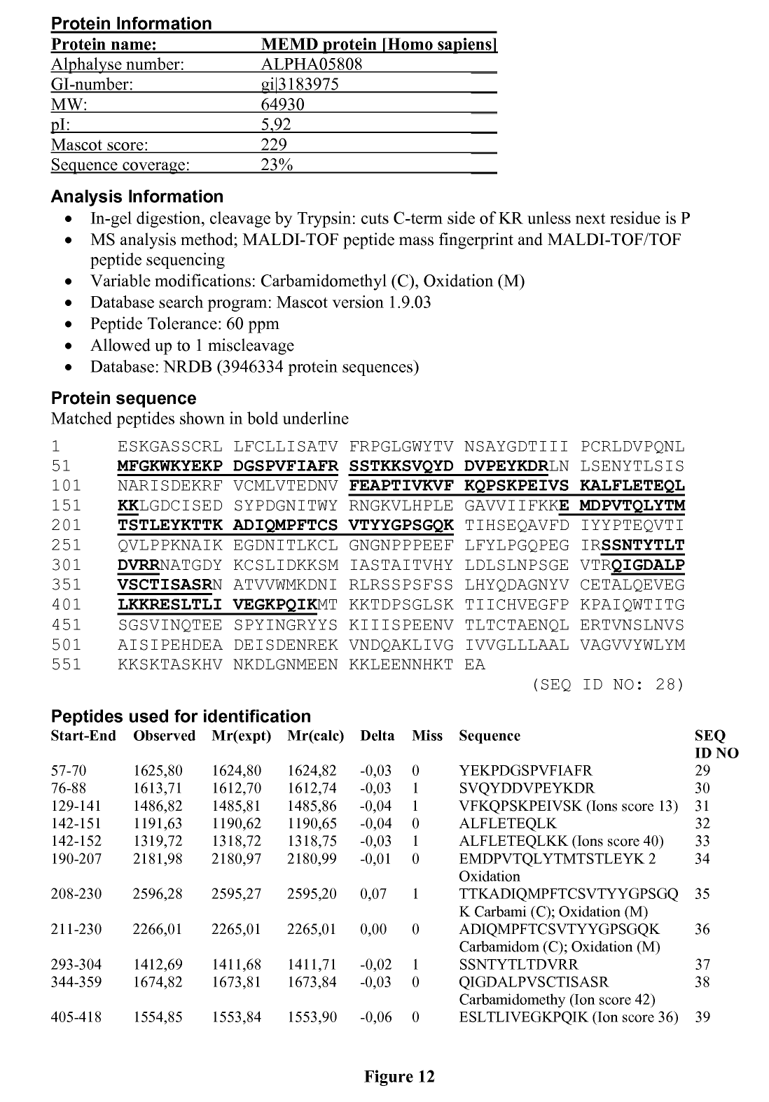

FIG. 12 shows the list of Example 10 of peptides used for mass fingerprinting and their mapped position in respect to the sequence on CD166 protein.

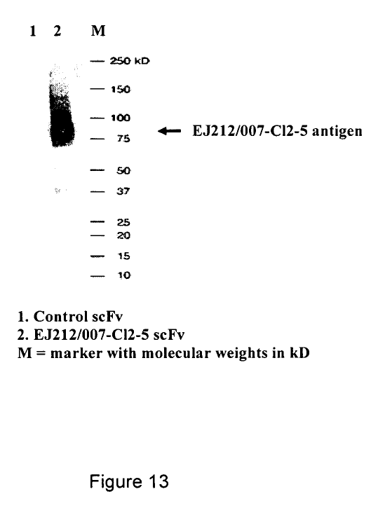

FIG. 13 shows the western blot analysis of Example 10 of the protein covalently linked with EJ212/007-Cl2-5 scFv from PM-1 cells.

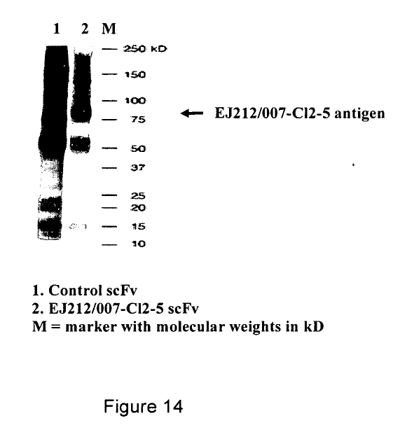

FIG. 14 shows the western blot analysis of Example 10 of the protein covalently linked with EJ212/007-Cl2-5 scFv from PM-1 cells. The indicated band for EJ212/007-Cl2-5 was excised and used for mass spectrometry analysis of the digested peptides.

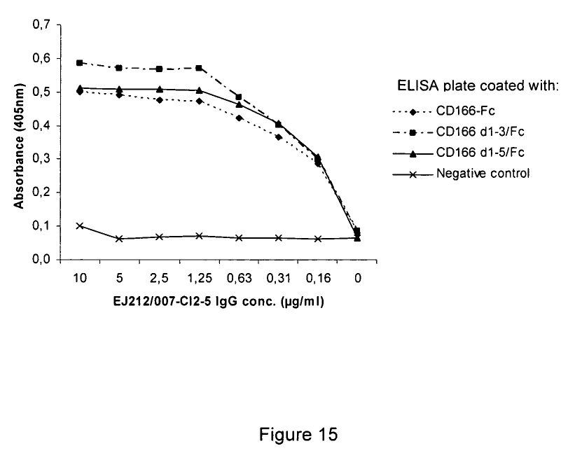

FIG. 15 shows the binding in ELISA of EJ212/007-Cl2-5 IgG to the human CD166 complete extracellular domain (CD166-Fc), extracellular domain 1-5 (CD166 d1-5/Fc) and extracellular domain 1-3 (CD166 d1-3/Fc) of Example 11. The extracellular CD166 domains were expressed as recombinant Fc chimera.

The following non-limiting examples are illustrative of the present invention:

### EXAMPLES

### Example 1

### Novel Antibody

Given the need for tumor specific antibodies, a human antibody has been identified which is reactive against breast carcinoma PM-1 tumor cells (provided by DNR, Department of Tumorbiology) and negative against PBL (peripheral blood lymphocytes) and granulocytes. The antibody can specifically bind to CD166. Single chain form of the antibody was cloned in the pHOG21 plasmid (at the NcoI and Not I restriction sites) which contains a c-myc and 6×His tag epitopes. E. coli cells, XL-1 blue, were transformed, selected on ampicillin plates and the scFv was expressed upon IPTG induction. Purified scFv was tested by cell ELISA for selective biological activity with PM-1 versus PBL and granulocytes. The selective biological activity was further confirmed by flow cytometry on the same cell types plus other breast carcinoma cell lines.

### Sequencing

The nucleotide sequences of the heavy and light chain of the antibody producing clone were sequenced. The antibody is designated as EJ212/007-Cl2-5 (scFv). The nucleotide sequence and amino acid sequence of the light and heavy chain of EJ212/007-Cl2-5 (scFv) are shown in FIG. 1. The CDR regions of the light and heavy chains of EJ212/007-Cl2-5 are shown in Table 1.

The IgG form of this antibody has also been made. The IgG form is of the IgG1 isotype and it comprises two heavy chains and two light chains. Each heavy chain comprises a V_{H} domain of SEQ ID NO: 9 and an IgG1 constant region. Each light chain comprises a V_{L} domain of SEQ ID NO: 10 and a kappa light constant region. The components of the IgG form of the antibody were cloned into vectors based on a vector published by Lars Norderhaug et al, JIM 204 (1997) 77-87 and using the method described in this citation. The vector contains a standard CMV promoter. An IgG leader sequence (mgwsciilflvatatgvhs) was introduced into each vector. The V_{H} and V_{L} domains were cloned into separate vectors containing genomic copies (introns+exons) of the human IgG1 and Kappa genes, respectively into Bsml and BSiWI sites which had been introduced by PCR. The IgG1 vector contains a neomycin resistance gene, whereas the Kappa vector contains a hygromycin resistance gene.

Further scFv antibodies which can specifically bind CD166 have also been generated.

### Example 2

### Specificity of EJ212/007-Cl2-5 by Flow Cytometry (FACS and Guava Easy Cyte)

To determine the tumor-specificity of the antibody, binding of EJ212/007-Cl2-5 was tested by flow cytometry (FACS, B&D and Guava EasyCyte, Guava Technologies) on PBLs and granulocytes and on breast carcinoma cell line MDA-MB231 (ATCC: HTB-26). Briefly, 1.2×10⁵ cells/100 μl were incubated with 10 μg/ml purified EJ212/007-Cl2-5 scFv or PBS. Bound scFv were detected with an anti-c-myc tag mouse monoclonal antibody (Invitrogen) followed by a FITC-labeled anti-mouse IgG (DAKO).

Flow cytometry results showed strong binding of EJ212/007-Cl2-5 to MDA-MB 231 (FIG. 2 A), but no or insignificant binding to PBL or granulocytes (FIGS. 2 B and C, respectively).

Binding to lung carcinoma cell line SW900 (ATCC: HTB-59) was also tested and FIG. 2D shows binding of EJ212/007-Cl2-5 to SW900 cells.

### Example 3

### Binding Affinity of EJ212/007-Cl2-5

Flow cytometry was used to assess the binding affinity of antibody EJ212/007-Cl2-5 to tumor cells. Increased concentrations of the scFv was tested against a fixed number of SW900 (ATCC: HTB-59) cells, a human lung cancer cell line, to establish a saturation curve. Bound scFv was detected as described above. The binding affinity expressed as the Kₘ was calculated by the Lineweaver-Burk method by plotting the inverse of the median fluorescence as a function of the inverse of the antibody molar concentration. The Kₘ was determined by the following equation: Kₘ=F_{Max}*a, where ‘a’ corresponds to the slope of the curve and F_{Max}=1/b was calculated from the plot (y=ax+b).

The binding curves and Lineweaver-Burk plots are shown in FIGS. 3 A and B and the Kₘ value was calculated as 2.1×10⁻⁸ M for the antibody EJ212/007-Cl2-5.

Binding affinity of EJ212/007-Cl2-5 Fab fragments to the CD166 antigen was measured by Biacore analysis. Recombinant ALCAM/Fc (250RU) was immobilized by amine coupling to a CM5 chip. A range of concentrations of the Fab antibody, from 3.9 to 125 nM (nanomolar), were run over the chip. The binding curves are shown in FIG. 3C. The binding affinity, expressed as the K_{d}, was calculated using the Heterogen ligand binding model in the Biacore T100 Evaluation software. The K_{d} value obtained for EJ212/007-Cl2-5 Fab was 2.9×10⁻⁸ M.

### Example 4

### Selectivity and Specificity of EJ212/007-Cl2-5 on Human Tissue Sections

The selectivity of EJ212/007-Cl2-5 scFv antibody was by determined by its binding to human tumor tissues using immunohistochemistry staining. The antibody was first tested against frozen SW900 cell line pellets to define the optimal conditions for staining. The antibody was then tested on a high-density array of human tumor tissues including breast, prostate and kidney from 11-14 different donors. The membranous staining intensity was estimated by visual inspection in a four step scale (0, 1, 2, 3). In addition, the fraction of tumor cells staining positive was estimated. A final IHC score was build from these two parameters according to Table 3.

The results are summarized in Table 4. Positivity for EJ212/007-Cl2-5 was found in 20 (51%) tumors.

The strongest binding was observed with, but not limited to breast, kidney and prostate with a highest score at 2+ and an overall percent of positive cells greater than 50%. In all cases, staining of the cytoplasm with 1+ score was observed.

Specificity of EJ212/007-Cl2-5 scFv antibody was determined by comparing the binding to a tissue microarray with 13 human breast tumor tissues and to a tissue microarray with 4 normal breast tissues using immunohistochemistry staining. Staining was performed as described above for selectivity testing.

EJ212/007-Cl2-5 scFv antibody showed clear specific membrane staining of 6/13 tumor tissues, whereas no specific membrane staining of normal human breast tissue was seen.

Specificity of EJ212/007-Cl2-5 IgG was tested on large tissue sections of 4 normal human brain and 4 ovary tissues, as well as 6 human brain tumor (glioblastoma) and 6 human ovarian tumor tissues. Staining was performed as described above for selectivity testing. In contrast to the scFv form, the IgG form of the antibody showed clear specific membrane staining of tumor breast tissue and normal human breast tissue.

The results for EJ212/007-Cl2-5 IgG are summarized in Table 5. EJ212/007-Cl2-5 IgG antibody showed clear specific membrane staining of 1/3 brain tumor tissues and 6/6 ovarian tumor tissues, whereas no specific membrane staining of normal brain and normal ovary tissue was seen.

The IgG antibody was also tested for cross-reactivity with mouse tissue. It showed cross-reactivity binding with normal tissue of mouse lung, prostate, stomach, whereas normal mouse brain, colon, heart, testis, liver and ovary showed no specific membrane staining.

### Example 5

### Antibody Dependent Cell Cytotoxicity (ADCC)

To investigate the ability of EJ212/007-Cl2-5 to induce ADCC, the antibody was cloned into IgG format and incubated with human tumor cells in the presence of freshly isolated human peripheral blood lymphocytes (PBL). The human tumor cells were selected from breast tumor cells (PM-1, MDA-MB 231 and BT474), lung tumor cells (SW900 and A-549) or prostate tumor cells (DU-145). The purpose of this test is to determine the usefulness of the antibody candidate for therapeutic application using the antibody as naked IgG.

In short, PM-1, MDA-MB 231, BT474, SW900, A-549 or DU-145 cells were harvested with trypsin/EDTA. After washing the cells once with RPMI/10% FCS and once with RPMI without serum, the tumor cells were labeled with calcein (calcein-acetomethylester, Invitrogen). 1.5×10⁴ labeled tumor cells were mixed with 3×10⁵ freshly isolated PBL and 1 μg/ml IgG antibody. In the assay an effector cell (PBL) to target cell (PM-1, MDA-MB 231, BT474, SW900, A-549 or DU-145) ratio of 20:1 was used, and all samples were tested in quadruplicates. Triton X-100 was added up to 0.8% in the maximum release samples. All samples were incubated for 4 hours at 37° C. in a humidified incubator with 5% CO₂. 100 μl supernatant was then analysed in a fluorescence plate reader (exitation wavelength 485 nm and emission wavelength 535 nm).

FIG. 4 shows that the addition of the EJ212/007-Cl2-5 IgG antibody to the tumor cells/PBL mixture resulted in approx. 20% killing of the tumor cells (PM-1 breast tumour cells). The addition of a negative control IgG antibody showed no killing, demonstrating the specificity of the killing induced by EJ212/007-Cl2-5 IgG. The numbers were calculated by subtraction of the background fluorescence (incubation of effector and target cells, without the addition of IgG antibody) and corrected for the fluorescence reduction (approx. 20%) in the maximum release samples that is seen due to the addition of Triton X-100.ADCC experiments with other tumor cell lines showed killing of approx. 10% of the lung tumor cells (SW900 and A-549), approx. 5 and 30% of the breast tumor cells (MDA-MB 231 and BT474, respectively) and approx. 3% of the prostate tumor cells (DU-145). Killing efficiency varied with each PBL donor.

### Example 6

### Growth Inhibition

The EJ212/007-Cl2-5 IgG was tested for growth inhibitory potential on CFSE-labeled MDA-MB 453 (ATCC: HTB-131) breast carcinoma cell line. CFSE has a bright fluorescence and is partitioned equally among daughter cells with each division. This allows differential staining of populations of cells. Populations in which cell division has been inhibited will exhibit higher fluorescence than populations which were allowed to divide freely. In other words, the inhibition of cell division causes a less marked reduction in fluorescence.

2×10⁶ cells were labeled with 0.625 μM CFSE for 15 minutes at 37° C. After washing the cells were divided over six T75 cell culture flasks and incubated at 37° C. and 5% CO₂ in the presence of 10 μg/ml EJ212/007-Cl2-5 IgG. Incubation without any antibody was used as a control. The cell culture medium was exchanged every other day with fresh medium and antibody. After 7 days of culture the cells were harvested with trypsine-EDTA, washed and transferred to a 96-well plate. After addition of Propidiium Iodide the fluorescence of the cells was measured using the Gauva EasyCyte.

FIG. 6 shows the mean fluorescence intensity after 7 days of culture. Cells that were incubated in the absence of an antibody (CFSE 453 cells) show a lower fluorescence intensity than the cells incubated with the EJ212/007-Cl2-5 IgG antibody. This shows that the cells divided less in the presence of the EJ212/007-Cl2-5 antibody and that the antibody has a growth inhibitory effect.

### Example 7

### Internalization

The EJ212/007-Cl2-5 scFv antibody was tested for internalization on PM-1 cells with the use of a CypHer5E-labeled secondary antibody. CypHer5E™ is a red-excitable, pH-sensitive cyanine dye derivative, which is minimally fluorescent at a basic pH and maximally fluorescent at an acidic pH. It is therefore ideally suited to report the movement of a receptor from the cell surface into acidic endosomes upon agonist stimulation. For the internalization assay 2×10⁵ cells were added per well of a black walled 96-well plate (Applied Biosystems). After addition of 2.5 μg/ml Cypher5E-labeled anti-c-myc IgG (9E10, Diatec) the cells were incubated for 30 minutes at 37° C. in the presence of 5 μg/ml EJ212/007-Cl2-5 scFv. As a control cells were incubated with a positive control antibody which is known to be internalized. After centrifugation for 3 minutes at 100 g the fluorescence inside the cells was measured with the 8200 Cellular Detection System (Applied Biosystems).

The CypHer5E-labeled secondary antibody was detected after incubation in the presence of EJ212/007-Cl2-5 scFv, indicating some internalization of the EJ212/007-Cl2-5 scFv (see FIG. 7A). FIG. 7B shows staining in the presence of the positive control antibody.

### Example 8

### Apoptosis

With the aim of evaluating the activity of the EJ212/007-Cl2-5 IgG antibody as an inductor of apoptosis we studied its effect on the BT474 (ATCC HTB-20) breast cancer cell line. For this purpose we investigated its activity (by means of Annexin V staining—Annexin V is labelled with FITC) alone and in combination with a well known protein kinase inhibitor and potent apoptosis inductor, Staurosporine. 2×10⁵ cells were plated in a 6-well plate 24 hours prior to addition of the drugs. Cells together with drugs were incubated for another 24 hours and the effect, after cell harvesting and Annexin V staining (100,000 cells), was measured by FACS analysis (EasyCyte). EJ212/007-Cl2-5 IgG antibody was tested at concentrations of 30 μg/ml 25, 20, 10 and 5 μg/ml in combination with 0.1 μM Staurosporine, and the EJ212/007-Cl2-5 IgG antibody alone at the highest concentration (30 μg/ml). The internal controls used were Staurosporine at 0.1 μM or DMSO (0.005%). To evaluate cell viability after incubation with the drugs, propidium iodide (1:4000; staining dead cells) was added at the final step of the Annexin V staining.

FIG. 8B shows that there is some induction of apoptosis by the EJ212/007-Cl2-5 IgG antibody alone and some induction by Staurosporine alone. The effect of EJ212/007-Cl2-5 IgG in combination with Staurosporine is greater than the combined effects of each individual agent, indicating a synergistic effect. Cell viability as shown in FIG. 8A is, as expected, inversely proportional to the induction of apoptosis.

### Example 9

### In Vivo Efficacy

To test the effect of the EJ212/007-Cl2-5 IgG on tumor cell growth in vivo 30 female nude (athymic nu/nu) mice were sub-cutaneously implanted with MDA-MB 231 tumor fragments and randomized into 2 treatment groups. Each group was treated intravenously via the tail with either vehicle control (PBS; 3 injections, every seventh day) or EJ212/007-Cl2-5 IgG (100 mg/kg body weight; 3 injections, every seventh day).

Tumors were measured three times a week. Individual mice were culled when the tumor reached the predetermined termination criterion of 4 grams (equal to 4000 mm³)

FIG. 9 shows the percentage of mice with tumor less than 4 grams after PBS or EJ212/007-Cl2-5 IgG injection. The group of animals treated with EJ212/007-Cl2-5 IgG showed reduced tumor growth compared to the group treated with PBS: the EJ212/007-Cl2-5 treated group had a higher percentage of mice with tumors less than 4 grams between 20 and 35 days after injection than the group treated with PBS.

### Example 10

### Characterization of the EJ212/007-Cl2-5 Antigen

Characterization of the antigen of the EJ212/007-Cl2-5 antibody was achieved using two different methods: immunoprecipitation and cross-linking

### Method 1: Immunoprecipitation

Membrane proteins were isolated from two EJ212/007-Cl2-5 antigen-positive cell lines (PM-1 and DU-145; determined by FACS analysis) and concentrated by ultra-centrifugation. The membranes were resuspended in detergent (CHAPS/DOC) which was then removed from the sample by using Detergent Removing Resin (Pierce) in column format. The purified membrane proteins before and after sample concentration, and the cytosolic fraction were subsequently analysed by SDS-PAGE/Western under non-reducing conditions. The blot was probed with the EJ212/007-Cl2-5 IgG and corresponding secondary anti-human IgG coupled to HRP in order to visualise the proteins by chemiluminescence.

The clear band (>62 KDa) in FIG. 10 shows that the EJ212/007-Cl2-5 antigen is present in the purified membrane sample.

Capture of the antigen was achieved by immunoprecipitation. The EJ212/007-Cl2-5 IgG was covalently linked to CarboLink™ beads (Pierce) through sugar groups on the Fc domain, while leaving the antigen-binding sites unobstructed. As a positive control an IgG towards a known antigen was linked to a separate sample of beads. The extracted membrane proteins were run over the beads in column format and the antigens that bound to the antibody were eluted and analysed by SDS-PAGE/Western under non-reducing conditions.

The first two lanes in FIG. 11 shows the bands of the captured positive control antigen. Lane 3 shows a band between 83 and 175 KDa of material that was eluted from the EJ212/007-Cl2-5-linked beads. This band was excised from a non-reducing SDS-PAGE gel after staining with SyproRuby and digested with trypsin for subsequent analysis of the extracted peptides by mass spectrometry.

All the peptides recovered and reconstructed to their right masses were used directly in a peptide mass fingerprinting step to obtain an ID for the protein. The list of peptides used for identification and their mapped positions in respect to the sequence of MEMD (gi|3183975), identical to ALCAM (activated leukocyte cell adhesion molecule; CD166), are shown in FIG. 12.

### Method 2: Cross-Linking

EJ212/007-Cl2-5 scFv (His-tagged) with a cysteine introduced by point mutation at position 68 (Kabat, RFCIS) was expressed in E. coli TG1 and purified in the presence of 1 mM dithiothreitol. The resulting scFv EJ212/007-Cl2-5 T68C was then incubated with an excess of the linker Mts-Atf-LC-Biotin (Pierce, Prod. No 33083), a three-armed linker with the following groups on each arm; biotin (on cleavable arm by reducing reagent), photoactivatable non-specific reactive group, and an amino-reactive group. The scFv with linker was then desalted and stored in PBS.

PM-1 cells (2×10⁶) were incubated with 25 μg of the above linker coupled scFv. After washing with PBS the cells were exposed to UV light at 302 nm. This was followed by lysis of the cells by NP-40 in the presence of DNAsel. After incubation on ice SDS was added and the samples were loaded on a His-column. The column was washed in the presence of NP-40 and SDS and the protein of interest was eluted with 5 mM DTT in the same detergents. The eluted sample was then mixed with streptavidin beads and the beads were washed in the presence of SDS. The SDS concentrations was increased and the beads were incubated at 90° C. for five minutes. The supernatant was then run on a 7% SDS-PAGE, transferred to a PVDF membrane and detected with streptavidin-HRP.

FIG. 13 shows a clear band >75 KDa that represent the EJ212/007-Cl2-5 antigen. The same experiment was repeated with 67×10⁶ and 75 μg of scFv with linker. A third of the sample eluted from the beads was run on a 7% SDS-PAGE and blotted on a PVDF membrane as described above (FIG. 14). The remaining sample was run on a 7% SDS-PAGE and stained with SyproRuby. The band of interest as indicated in FIG. 14 was cut out from the gel and sent for mass spectography analysis (Table 6). Relevant peptides identified in the mass spectography report were assigned to ALCAM (CD166).

### Example 11

### Binding of EJ212/007-Cl2-5 to the Identified Antigen

Two constructs consisting of either domain 1-3 (CD166 d1-3/Fc) or domain 1-5 (CD166 d1-5/Fc) of the CD166 antigen were cloned in the PLNO-Fc-Hyg-IgG vector. Each construct was expressed in HEK cells by transfection. After four days of expression the media was harvested and the CD166 d1-3/Fc and CD166 d1-5/Fc were purified with affinity chromatography, protein A, and dialysed overnight in PBS.

Binding of the EJ212/007-Cl2-5 IgG to the CD166 antigen was tested in ELISA. An ELISA plate was coated overnight with 10 μg/ml of a commercially available recombinant CD166-Fc antigen (R&D systems), CD166 d1-5/Fc or CD166 d1-3/Fc. An IgG with irrelevant binding specificity was added to the ELISA as a negative control. After washing with PBS/0.05% Tween the plate was incubated for 1 hour with a dilution series of EJ212/007-Cl2-5 IgG ranging from 10 to 0.16 μg/ml. Detection of the bound IgG was done by incubation for 1 hour with 1 μg/ml anti-kappa-HRP and 15 minutes with the 1-step ABTS substrate (Pierce). The absorbance was measured at 405 nm.

FIG. 15 shows binding of EJ212/007-Cl2-5 IgG to recombinant CD166-Fc, CD166 d1-5/Fc and CD166 d1-3/Fc, indicating that the epitope of EJ212/007-Cl2-5 scFv is located on domain 1-3 of the CD166 antigen.

Binding of EJ212/007-Cl2-5 IgG to mouse recombinant CD166-Fc (R&D systems) was shown in a similar ELISA as described above for human CD166-Fc.

TABLE 1

| | | |
|---|---|---|
| SEQ ID NO | Description | Sequence |

| | | |
|---|---|---|
| 1 | EJ212/007-CI2-5, | FIG. 1 |
| | na | |
| 2 | EJ212/007-CI2-5, | FIG. 1 |
| | pp | |
| 3 | EJ212/007-CI2-5, | SYAMS |
| | CDR1 VH pp | |
| 4 | EJ212/007-CI2-5, | AISGSGGSTYYADSVKG |
| | CDR2 VH pp | |
| 5 | EJ212/007-CI2-5, | GGGVVEF |
| | CDR3 VH pp | |
| 6 | EJ212/007-CI2-5, | RASQDISSYFA |
| | CDR1 VL pp | |
| 7 | EJ212/007-CI2-5, | AASTLRS |
| | CDR2 VL pp | |
| 8 | EJ212/007-CI2-5, | QQSYSTPRIT |
| | CDR3 VL pp | |
| 9 | EJ212/007-CI2-5, | EVQLLESGGGLVQPGGSLRLSCAASGFTFSSYAMSWVR |
| | V_{H} pp | QAPGKGLEWVSAISGSGGSTYYADSVKGRFTISRDNSKN |
| | | TLYLQMNSLRAEDTAVYYCARGGGVVEFWGQGTLVTVSS |
| 10 | EJ212/007-CI2-5, | DIRMTQSPSFLSASVGDRVTITCRASQDISSYFAWYQQKP |
| | V_{L} pp | GKAPKLLIYAASTLRSGVPSRFSGSGSGTDFTLTISSLQPE |
| | | DFATYYCQQSYSTPRITFGQGTRLEIK |
| 11 | IgG leader pp | MGWSCIILFLVATATGVHS |
| 12 | IgG1 constant | ASTKGPSVFPLAPSSKSTSGGTAALGCLVKDYFPEPVTVS |
| | region pp | WNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPS |
| | | RDELTKNQVSLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |
| 13 | Kappa pp | RTVAAPSVFIFPPSDEQLKSGTASVVCLLNNFYPREAKVQ |
| | | WKVDNALQSGNSQESVTEQD |
| | | SKDSTYSLSSTLTLSKADYEKHKVYACEVTHQGLSSPVTK |
| | | SFNRGEC |
| 14 | EJ212/007-CI2-5, | GAGGTGCAGCTGTTGGAGTCCGGGGGAGGCTTAGTTC |
| | V_{H} na | AGCCTGGGGGGTCCCTGAGACTCTCCTGTGCAGCCTC |
| | | TGGATTCACCTTTAGCAGCTATGCCATGAGCTGGGTCC |
| | | GCCAGGCTCCAGGGAAGGGGCTGGAGTGGGTCTCAGC |
| | | TATTAGTGGTAGTGGTGGTAGTACATACTACGCAGACT |
| | | CCGTGAAGGGCCGGTTCACCATCTCCAGAGACAATTCC |
| | | AAGAACACGCTGTATCTGCAAATGAACAGCCTGAGAGC |
| | | CGAGGACACGGCCGTATATTACTGTGCGAGGGGGGGA |
| | | GGAGTGGTCGAATTTTGGGGCCAGGGAACCCTGGTCA |
| | | CTGTCTCCTCA (see FIG. 1) |
| 15 | EJ212/007-CI2-5, | GACATCCGGATGACCCAGTCTCCATCCTTCCTGTCTGC |
| | V_{L} na | ATCTGTAGGAGACAGAGTCACCATCACTTGCCGGGCCA |
| | | GTCAGGACATTAGCAGTTATTTCGCCTGGTATCAGCAA |
| | | AAACCAGGGAAAGCCCCTAAGCTCCTGATCTATGCTGC |
| | | ATCCACTTTGCGAAGTGGGGTCCCATCAAGGTTCAGCG |
| | | GCAG |
| | | TGGATCTGGGACAGATTTCACTCTCACCATCAGCAGTC |
| | | TGCAACCTGAAGATTTTGCAACTTACTACTGTCAACAGA |
| | | GTTACAGTACCCCTCGGATCACCTTCGGCCAAGGGACA |
| | | CGACTGGAGATTAAA (see FIG. 1) |

TABLE 2

| VH | Framework 1 | Framework 2 |
|---|---|---|
| EJ212/007-CI2-5 | EVQLLESGGGLVQPGGSLRLSCAASGFTFS | WVRQAPGKGLEWVS |
| VH | Framework 3 | Framework 4 |
| EJ212/007-C12-5 | RFTISRDNSKNTLYLQMNSLRAEDTAVYYCAR | WGQGTLVTVSS |
| VL | Framework 1 | Framework 2 |
| EJ212/007-C12-5 | DIRMTQSPSFLSASVGDRVTITC | WYQQKPGKAPKLLIY |
| VL | Framework 3 | Framework 4 |
| EJ212/007-C12-5 | GVPSRFSGSGSGTDFTLTISSLQPEDFATYYC | FGQGTRLEIK |

TABLE 3
IHC scoring

| Score | Definition |
|---|---|
| Negative | Int* 0 |
| Weak | Int 1 |
| | Int 2 in ≦70% of tumor cells |
| | Int 3 in ≦30% of tumor cells |
| Strong | Int 2 in >70% of tumor cells |
| | Int 3 in >30% of tumor cells |

*Int = staining intensity.

TABLE 4
High density tumor array for EJ212/007-Cl2-5 scFv

| Tissue | Membrane staining | Score Range |
|---|---|---|
| Breast | 6/13 | weak (3) and strong (3) |
| Prostate | 9/11 | weak (6) and strong (3) |
| Kidney | 5/14 | weak (3) and strong (2) |

Numbers in parentheses indicate the number of patients showing 2+ scoring

TABLE 5
Membrane staining of large tissues sections of normal human tissues
and corresponding tumor tissues with EJ212/007-Cl2-5 IgG

| Tissue | Normal | Tumor |
|---|---|---|
| Brain | 0/4 | 1/3 |
| Ovary | 0/4 | 6/6 |

TABLE 6

| | Query | | | |
|---|---|---|---|---|
| | 308 | 393 | 395 | 403 |
| Observed | 512.4489 | 897.9930 | 901.3573 | 755.0889 |
| Mr (expt) | 1534.3248 | 2690.9572 | 2701.0501 | 3016.3266 |
| Mr (calc) | 1533.9283 | 2690.3585 | 2700.3006 | 3015.5593 |
| Delta | 0.3966 | 0.5986 | 0.7495 | 0.7673 |
| Miss | 0 | 0 | 0 | 0 |
| Score | 64 | 103 | 65 | 65 |
| Expect | 0.00022 | 1.5e-08 | 9.4e-05 | 0.00011 |
| Rank | 1 | 1 | 1 | 1 |
| Peptide | K.VLHPLEGA | K.SMIASTAITV | K.CLGNGNPPP | K.TIHSEQAVF |
| | VVIIFK.K | HYLDLSLNPSG | EEFLFYLPGQP | DIYYPTEQVT |
| | | EVTR.Q + | EGIR.S | IQVLPPK.N |
| | | Oxidation (M) | | |

1. A binding protein which comprises at least one light chain variable (VL) region that comprises three CDRs, wherein said light chain variable region comprises:
(i) a VL CDR1 that comprises the amino acid sequence of SEQ ID NO: 6,
(ii) a VL CDR2 that comprises the amino acid sequence of SEQ ID NO: 7, and
(iii) a VL CDR3 that comprises the amino acid sequence of SEQ ID NO: 8.

2. A binding protein according to claim 1, which additionally comprises at least one heavy chain variable (VH) region that comprises at least one heavy chain CDR, wherein said heavy chain CDR is selected from the group consisting of:
(i) a VH CDR1 that comprises the amino acid sequence of SEQ ID NO: 3,
(ii) a VH CDR2 that comprises the amino acid sequence of SEQ ID NO: 4, and
(iii) a VH CDR3 that comprises the amino acid sequence of SEQ ID NO: 5.

3. A binding protein according to claim 1, which additionally comprises at least one heavy chain variable (VH) region that comprises at least two heavy chain CDRs, wherein said heavy chain CDRs are selected from the group consisting of:
(i) a VH CDR1 that comprises the amino acid sequence of SEQ ID NO: 3,
(ii) a VH CDR2 that comprises the amino acid sequence of SEQ ID NO: 4, and
(iii) a VH CDR3 that comprises the amino acid sequence of SEQ ID NO: 5.

4. A binding protein according to claim 1, which additionally comprises at least one heavy chain variable (VH) region, wherein said heavy chain variable region comprises at least one of each of the following heavy chain CDRs:
(i) a VH CDR1 that comprises the amino acid sequence of SEQ ID NO: 3,
(ii) a VH CDR2 that comprises the amino acid sequence of SEQ ID NO: 4, and
(iii) a VH CDR3 that comprises the amino acid sequence of SEQ ID NO: 5.

5. A binding protein according to claim 1, wherein said light chain variable region comprises the amino acid sequence given in SEQ ID NO: 10.

6. A binding protein according to claim 1, which additionally comprises at least one heavy chain variable region that comprises the amino acid sequence of SEQ ID NO: 9.

7. A binding protein according to claim 1, wherein said light chain variable region comprises the amino acid sequence of SEQ ID NO: 10, and wherein said binding protein additionally comprises at least one heavy chain variable region that comprises the amino acid sequence of SEQ ID NO: 9.

8. A binding protein according to claim 1 comprising the amino acid sequence of SEQ ID NO: 2, or a fragment thereof.

9. A binding protein as claimed in claim 1, wherein the binding protein is an antibody or antibody fragment, or the binding protein comprises an antibody or antibody fragment.

10. A binding protein as claimed in claim 9, wherein the binding protein is a human antibody or human antibody fragment, or the binding protein comprises a human antibody or a human antibody fragment.

11. A binding protein as claimed in claim 9, wherein the antibody is a whole antibody (preferably an IgG, IgA, IgE, IgM, or IgD), monoclonal antibody, polyclonal antibody, a humanized antibody or a chimeric antibody.

12. A binding protein as claimed in claim 9, wherein the antibody fragment is a Fab, Fab′, F(ab′)2, scFv, Fv, dsFv, ds-scFv, Fd, dAbs, T and Abs dimer, minibody, diabody, or multimers thereof, or a bispecific antibody fragment.

13. A binding protein as claimed in claim 9, wherein the antibody or antibody fragment comprises all or a portion of a heavy chain constant region and/or all or a portion of a kappa or lambda light chain constant region.

14. A binding protein as claimed in claim 1, which is tumor specific.

15. A binding protein as claimed in claim 14, wherein the binding protein binds to one or more types of tumor cell or sample.

16. A binding protein as claimed in claim 14, wherein the binding protein binds to melanoma, prostate cancer, breast cancer, colorectal cancer, bladder cancer, ovarian cancer, pancreas cancer, lung cancer, esophageal squamous cell carcinoma, brain cancer and/or kidney cancer tissue.

17. A binding protein as claimed in claim 14, wherein the binding protein binds to one or more types of breast cancer cell or sample.

18. A binding protein as claimed in claim 1, wherein the binding protein can bind to CD166.

19. A binding protein as claimed in claim 14, wherein the binding protein does not significantly bind to peripheral blood lymphocytes (PBLs) and/or does not significantly bind to granulocytes.

20. A binding protein as claimed in claim 14, wherein the binding protein shows a measurable or significant binding to breast cancer cell line MDA-MB 231 but shows insignificant or unmeasurable binding to granulocytes or peripheral blood lymphocytes (PBLs).

21. A binding protein as claimed in claim 1, wherein the binding protein has a binding affinity for one or more types of cancer cells, preferably a breast cancer cell, prostrate cancer cell and/or lung cancer cell, which corresponds to a Kₘ of less than 1 μM, more preferably of less than 500, 400 or 300 nM, even more preferably of less than 200, 190, 180, 170, 160, 150, 140, 130, 120, 110, or 100 nM, most preferably of less than 90, 80, 70, 60, 50, 40, 30, 20, 10, 5 or 1 nM.

22. A binding protein as claimed in claim 21, wherein the binding protein has a binding affinity of 2.9×10⁻⁸ M or less, or 2.1×10⁻⁸ M or less.

23. A binding protein as claimed in claim 1, wherein the binding protein has a Kₘ for one or more types of cancer cells, preferably a breast cancer cell, prostrate cancer cell and/or lung cancer cell, which is at least 1, more preferably at least 2, 3, 4 or 5 orders of magnitude lower than the Kₘ for one or more types of non-cancerous or normal cells, preferably peripheral blood lymphocytes and/or granulocytes, when the binding affinity is assayed under comparable conditions.

24. A binding protein capable of binding an antigen on a tumor cell, preferably a breast cancer cell, wherein the binding protein can be identified by a method comprising:
(1) incubating a fixed number of tumor cells, preferably breast cancer cells, with a minimal concentration of a binding protein as claimed in claim 1, preferably an antibody or antibody fragment (Ab1) that generates maximal binding against the fixed number of tumor cells and measuring median fluorescence of Ab1 (MF_{Ab1});
(2) testing two or more concentrations of a test binding protein (Ab2) by adding Ab2 to the Ab1 and tumor cells, and measuring median fluorescence (MF_{(Ab1+Ab2)});
(3) measuring background median fluorescence (MF_{Bgd});
(4) calculating PI, wherein
PI=[(MF_{(Ab1+Ab2)}−MF_{Bgd})/(MF_{Ab1}−MF_{Bgd})]×100; and
(5) comparing the PI to a control PI value;
wherein, a PI that has a statistically significant difference from the control PI indicates that the test binding protein is capable of binding the antigen on the tumor cell.

25. A binding protein as claimed in claim 1, wherein the binding protein is a human protein.

26. A binding protein as claimed in claim 1, to which is attached one or more radiotherapeutic agent, anti-angiogenic agent, apoptosis-inducing agent, anti-tubulin drug, anti-cellular, cytotoxic agent or coagulant.

27. A binding protein as claimed in claim 1 which is labelled with a detectable marker.

28. A fusion protein or conjugate comprising a binding protein as claimed in claim 1 fused or conjugated to a cell binding protein such as an immunoglobulin, hormone, growth factor, lectin, insulin, low density lipoprotein, glucagon, endorphin, transferrin, bombesin, asialoglycoprotein, glutathione-S-transferase (GST), haemagglutinin (HA) or truncated myc.

29. A fusion protein or conjugate as claimed in claim 28, to which is attached one or more radiotherapeutic agent, anti-angiogenic agent, apoptosis-inducing agent, anti-tubulin drug, anti-cellular, cytotoxic agent or coagulant.

30. A fusion protein or conjugate as claimed in claim 28 which is labelled with a detectable marker.

31. A diagnostic or imaging agent comprising a binding protein as claimed in claim 1 which is labelled with a detectable marker.

32. A diagnostic or imaging agent comprising a fusion protein or conjugate as claimed in claim 28 which is labelled with a detectable marker.

33. A nucleic acid molecule comprising a nucleotide sequence which encodes a binding protein, as claimed in claim 1.

34. A nucleic acid molecule comprising a nucleotide sequence which encodes a fusion protein or conjugate as claimed in claim 28.

35. A nucleic acid molecule as claimed in claim 33 comprising the nucleotide sequence given in SEQ ID NO: 1.

36. A nucleic acid molecule as claimed in claim 34 comprising the nucleotide sequence given in SEQ ID NO: 1.

37. A vector, preferably a recombinant expression vector, comprising a nucleic acid molecule as claimed in claim 33.

38. A vector, preferably a recombinant expression vector, comprising a nucleic acid molecule as claimed in claim 34.

39. A host cell comprising a vector as claimed in claim 37.

40. A host cell comprising a vector as claimed in claim 38.

41. A host cell expressing a binding protein as claimed in claim 1.

42. A host cell expressing a fusion protein or conjugate as claimed in claim 28.

43. A transgenic non-human animal comprising a nucleic acid molecule as claimed in claim 33.

44. A transgenic non-human animal comprising a nucleic acid molecule as claimed in claim 34.

45. A transgenic non-human animal comprising a vector as claimed in claim 37.

46. A transgenic non-human animal comprising a vector as claimed in claim 38.

47. A composition comprising a binding protein as claimed in claim 1, optionally additionally comprising one or more pharmaceutically acceptable excipients, carriers, diluents, buffers or stabilizers.

48. A composition comprising a fusion protein or conjugate as claimed in claim 28, optionally additionally comprising one or more pharmaceutically acceptable excipients, carriers, diluents, buffers or stabilizers.

49. A method of treating a subject comprising administering an effective amount of a binding protein as claimed in claim 1, to the subject, or to a sample removed from the subject and which is subsequently returned to the subject.

50. A method as claimed in claim 49, wherein the subject is a mammal, preferably a human.

51. A method as claimed in claim 49, wherein the subject has melanoma, prostate cancer, breast cancer, colorectal cancer, bladder cancer, ovarian cancer, pancreas cancer, lung cancer, esophageal squamous cell carcinoma, brain cancer and/or kidney cancer.

52. A method of treating a subject comprising administering an effective amount of a fusion protein or conjugate as claimed in claim 28, to the subject, or to a sample removed from the subject and which is subsequently returned to the subject.

53. A method as claimed in claim 52, wherein the subject is a mammal, preferably a human.

54. A method as claimed in claim 52, wherein the subject has melanoma, prostate cancer, breast cancer, colorectal cancer, bladder cancer, ovarian cancer, pancreas cancer, lung cancer, esophageal squamous cell carcinoma, brain cancer and/or kidney cancer.

55. A method of diagnosis of a disease or disorder in a subject comprising the administration of an appropriate amount of a binding protein as claimed in claim 1 to the subject and detecting the presence and/or amount and/or the location of the binding protein in the subject.

56. A method as claimed in claim 55, wherein the disease or disorder is cancer, preferably melanoma, prostate cancer, breast cancer, colorectal cancer, bladder cancer, ovarian cancer, pancreas cancer, lung cancer, esophageal squamous cell carcinoma, brain cancer and/or kidney cancer.

57. A method of diagnosis of a disease or disorder in a subject comprising the administration of an appropriate amount of a fusion protein or conjugate as claimed in claim 28 and detecting the presence and/or amount and/or the location of the fusion protein or conjugate in the subject.

58. A method as claimed in claim 57, wherein the disease or disorder is cancer, preferably melanoma, prostate cancer, breast cancer, colorectal cancer, bladder cancer, ovarian cancer, pancreas cancer, lung cancer, esophageal squamous cell carcinoma, brain cancer and/or kidney cancer.

59. A method of diagnosing a disease, preferably cancer, in a mammal comprising the step of:
(1) contacting a test sample taken from said mammal with a binding protein as claimed in claim 1.

60. A method as claimed in claim 59 which additionally comprises the steps:
(2) measuring the presence and/or amount and/or location of a complex formed between the binding proteins and an antigen in the test sample; and, optionally
(3) comparing the presence and/or amount of binding protein-antigen complex in the test sample to a control.

61. A method of diagnosing a disease, preferably cancer, in a mammal comprising the step of:
(1) contacting a test sample taken from said mammal with any one or more of the fusion proteins or conjugates as claimed in claim 28.

62. A method as claimed in claim 61 which additionally comprises the steps:
(2) measuring the presence and/or amount and/or location of a complex formed between the fusion proteins or conjugates and an antigen in the test sample; and, optionally
(3) comparing the presence and/or amount of fusion protein- or conjugate-antigen complex in the test sample to a control.

63. A method of imaging of a subject comprising administering an appropriate amount of a binding protein as claimed in claim 1 to the subject and detecting the presence and/or amount and/or the location of the binding protein in the subject.

64. A method of imaging of a subject comprising administering an appropriate amount of a fusion protein or conjugate as claimed in claim 28 to the subject and detecting the presence and/or amount and/or the location of the fusion protein or conjugate in the subject.

65. A method of producing a binding protein, as claimed in claim 1, comprising a step of culturing a host cell comprising a vector, preferably a recombinant expression vector, comprising a nucleic acid molecule comprising a nucleotide sequence which encodes a binding protein, as claimed in claim 1.

66. A method of producing a fusion protein or conjugate, as claimed in claim 28, comprising a step of culturing a host cell comprising a vector, preferably a recombinant expression vector, comprising a nucleic acid molecule comprising a nucleotide sequence which encodes fusion protein or conjugate, as claimed in claim 28.

67. A method of producing a binding protein as claimed in claim 1, comprising the steps of:
(i) culturing a host cell comprising one or more of the recombinant expression vectors or one or more of nucleic acid molecules comprising a nucleotide sequence which encodes a binding protein, as claimed in claim 1, under conditions suitable for the expression of the protein; and optionally
(ii) isolating the binding protein from the host cell or from the growth medium/supernatant.

68. A method as claimed in claim 67, which additionally comprises the step of purifying the binding protein and/or formulating the product into a composition including at least one additional component, such as a pharmaceutically acceptable carrier, diluent or excipient.

69. A method of producing a fusion protein or conjugate
as claimed in claim 28, comprising the steps of:
(i) culturing a host cell comprising one or more of the recombinant expression vectors or one or more of nucleic acid molecules comprising a nucleotide sequence which encodes a fusion protein or conjugate, as claimed in claim 28, under conditions suitable for the expression of the fusion protein or conjugate; and optionally
(ii) isolating the fusion protein or conjugate from the host cell or from the growth medium/supernatant.

70. A method as claimed in claim 69, which additionally comprises the step of purifying the binding protein and/or formulating the product into a composition including at least one additional component, such as a pharmaceutically acceptable carrier, diluent or excipient.

71. The composition as claimed in claim 47, wherein said composition further comprises an additional active agent.

72. The composition as claimed in claim 71, wherein said additional active agent is a pro-apoptotic agent.

73. The composition as claimed in claim 71, wherein said additional active agent is one or more selected from the group consisting of antibodies which bind to other targets, cytokines, antimetabolites, alkylating agents, purine analogs and related inhibitors, pyrimidine analogs, folic acid analogs, vinca alkaloids, epipodopyllotoxins, antibiotics, L-aspaginase, estrogens, antiestrogens, androgens, antiandrogens, adrenocorticosteroides, progestines, adrenocortical suppressant, gonadotropin releasing hormone analogs, interferons, topoisomerase inhibitor, anthracenedione substituted urea, methyl hydrazine derivatives, platinum coordination complexes or chemical agents, and drugs controlling side effects.

74. The composition as claimed in claim 72, wherein said pro-apoptotic agent is staurosporine.


SEQUENCE LISTING (43 sequences)

SEQ ID NO: 1 - DNA, 735 bp, artificial sequence
  single chain variable fragment antibody
gaggtgcagctgttggagtccgggggaggcttagttcagcctggggggtccctgagactctcctgtgcagcctctggattcacctttagcagctatgccatgagctgggtccgccaggctccagggaaggggctggagtgggtctcagctattagtggtagtggtggtagtacatactacgcagactccgtgaagggccggttcaccatctccagagacaattccaagaacacgctgtatctgcaaatgaacagcctgagagccgaggacacggccgtatattactgtgcgagggggggaggagtggtcgaattttggggccagggaaccctggtcactgtctcctcaaagctttcagggagtgcatccgccccaaaacttgaagaaggtgaattttcagaagcacgcgtagacatccggatgacccagtctccatccttcctgtctgcatctgtaggagacagagtcaccatcacttgccgggccagtcaggacattagcagttatttcgcctggtatcagcaaaaaccagggaaagcccctaagctcctgatctatgctgcatccactttgcgaagtggggtcccatcaaggttcagcggcagtggatctgggacagatttcactctcaccatcagcagtctgcaacctgaagattttgcaacttactactgtcaacagagttacagtacccctcggatcaccttcggccaagggacacgactggagattaaa

SEQ ID NO: 2 - protein, 245 aa, artificial sequence
  single chain variable fragment antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGGVVEFWGQGTLVTVSSKLSGSASAPKLEEGEFSEARVDIRMTQSPSFLSASVGDRVTITCRASQDISSYFAWYQQKPGKAPKLLIYAASTLRSGVPSRFSGSGSGTDFTLTISSLQPEDFATYYCQQSYSTPRITFGQGTRLEIK

SEQ ID NO: 3 - protein, 5 aa, artificial sequence
  complementarity determining region 1 of heavy chain
SYAMS

SEQ ID NO: 4 - protein, 17 aa, artificial sequence
  complementarity determining region 2 of heavy chain
AISGSGGSTYYADSVKG

SEQ ID NO: 5 - protein, 7 aa, artificial sequence
  complementarity determining region 3 of heavy chain
GGGVVEF

SEQ ID NO: 6 - protein, 11 aa, artificial sequence
  complementarity determining region 1 of light chain
RASQDISSYFA

SEQ ID NO: 7 - protein, 7 aa, artificial sequence
  complementarity determining region 2 of light chain
AASTLRS

SEQ ID NO: 8 - protein, 10 aa, artificial sequence
  complementarity determining region 3 of light chain
QQSYSTPRIT

SEQ ID NO: 9 - protein, 116 aa, artificial sequence
  heavy chain of antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGGVVEFWGQGTLVTVSS

SEQ ID NO: 10 - protein, 108 aa, artificial sequence
  light chain of antibody
DIRMTQSPSFLSASVGDRVTITCRASQDISSYFAWYQQKPGKAPKLLIYAASTLRSGVPSRFSGSGSGTDFTLTISSLQPEDFATYYCQQSYSTPRITFGQGTRLEIK

SEQ ID NO: 11 - protein, 19 aa, Homo sapiens
MGWSCIILFLVATATGVHS

SEQ ID NO: 12 - protein, 330 aa, Homo sapiens [sequence omitted]

SEQ ID NO: 13 - protein, 107 aa, Homo sapiens [sequence omitted]

SEQ ID NO: 14 - DNA, 348 bp, artificial sequence
  variable heavy chain
gaggtgcagctgttggagtccgggggaggcttagttcagcctggggggtccctgagactctcctgtgcagcctctggattcacctttagcagctatgccatgagctgggtccgccaggctccagggaaggggctggagtgggtctcagctattagtggtagtggtggtagtacatactacgcagactccgtgaagggccggttcaccatctccagagacaattccaagaacacgctgtatctgcaaatgaacagcctgagagccgaggacacggccgtatattactgtgcgagggggggaggagtggtcgaattttggggccagggaaccctggtcactgtctcctca

SEQ ID NO: 15 - DNA, 324 bp, artificial sequence
  variable light chain
gacatccggatgacccagtctccatccttcctgtctgcatctgtaggagacagagtcaccatcacttgccgggccagtcaggacattagcagttatttcgcctggtatcagcaaaaaccagggaaagcccctaagctcctgatctatgctgcatccactttgcgaagtggggtcccatcaaggttcagcggcagtggatctgggacagatttcactctcaccatcagcagtctgcaacctgaagattttgcaacttactactgtcaacagagttacagtacccctcggatcaccttcggccaagggacacgactggagattaaa

SEQ ID NO: 16 - protein, 30 aa, artificial sequence
  framework region 1 of heavy chain
EVQLLESGGGLVQPGGSLRLSCAASGFTFS

SEQ ID NO: 17 - protein, 14 aa, artificial sequence
  framework region 2  of heavy chain
WVRQAPGKGLEWVS

SEQ ID NO: 18 - protein, 32 aa, artificial sequence
  framework region 3 of heavy chain
RFTISRDNSKNTLYLQMNSLRAEDTAVYYCAR

SEQ ID NO: 19 - protein, 11 aa, artificial sequence
  framework region 4 of heavy chain
WGQGTLVTVSS

SEQ ID NO: 20 - protein, 23 aa, artificial sequence
  framework region 1 of light chain
DIRMTQSPSFLSASVGDRVTITC

SEQ ID NO: 21 - protein, 15 aa, artificial sequence
  framework region 2 of light chain
WYQQKPGKAPKLLIY

SEQ ID NO: 22 - protein, 32 aa, artificial sequence
  framework region 3 of light chain
GVPSRFSGSGSGTDFTLTISSLQPEDFATYYC

SEQ ID NO: 23 - protein, 10 aa, artificial sequence
  framework region 4 of light chain
FGQGTRLEIK

SEQ ID NO: 24 - protein, 16 aa, artificial sequence
  single chain variable fragment antibody peptide
KVLHPLEGAVVIIFKK

SEQ ID NO: 25 - protein, 27 aa, artificial sequence
  single chain variable fragment antibody peptide
KSMIASTAITVHYLDLSLNPSGEVTRQ

SEQ ID NO: 26 - protein, 26 aa, artificial sequence
  single chain variable fragment antibody peptide
KCLGNGNPPPEEFLFYLPGQPEGIRS

SEQ ID NO: 27 - protein, 28 aa, artificial sequence
  single chain variable fragment antibody peptide
KTIHSEQAVFDIYYPTEQVTIQVLPPKN

SEQ ID NO: 28 - protein, 582 aa, Homo sapiens
ESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 29 - protein, 14 aa, Homo sapiens
YEKPDGSPVFIAFR

SEQ ID NO: 30 - protein, 13 aa, Homo sapiens
SVQYDDVPEYKDR

SEQ ID NO: 31 - protein, 13 aa, Homo sapiens
VFKQPSKPEIVSK

SEQ ID NO: 32 - protein, 10 aa, Homo sapiens
ALFLETEQLK

SEQ ID NO: 33 - protein, 11 aa, Homo sapiens
ALFLETEQLKK

SEQ ID NO: 34 - protein, 18 aa, Homo sapiens
EMDPVTQLYTMTSTLEYK

SEQ ID NO: 35 - protein, 23 aa, Homo sapiens
TTKADIQMPFTCSVTYYGPSGQK

SEQ ID NO: 36 - protein, 20 aa, Homo sapiens
ADIQMPFTCSVTYYGPSGQK

SEQ ID NO: 37 - protein, 12 aa, Homo sapiens
SSNTYTLTDVRR

SEQ ID NO: 38 - protein, 16 aa, Homo sapiens
QIGDALPVSCTISASR

SEQ ID NO: 39 - protein, 14 aa, Homo sapiens
ESLTLIVEGKPQIK

SEQ ID NO: 40 - DNA, 78 bp, Homo sapiens
gcggccgctggatccgaacaaaagctgatctcagaagaagacctaaactcacatcaccatcaccatcactaatctaga

SEQ ID NO: 41 - protein, 23 aa, Homo sapiens
AAAGSEQKLISEEDLNSHHHHHH

SEQ ID NO: 42 - protein, 19 aa, Homo sapiens
MGWSCIILFLVATATGVHS

SEQ ID NO: 43 - protein, 5 aa, Homo sapiens
RFCIS